(19) United States
(12) Patent Application Publication
(10) Pub. No.: US 2026/0306445 A1
(43) Pub. Date: Oct. 8, 2026

(54) COMPOSITION FOR USE IN THE PREVENTION AND/OR TREATMENT OF DISEASES MEDIATED BY TLR4, IL1R, COX1/2 AND/OR RBP4

(71) Applicants: SERVIZO GALEGO DE SAUDE (SERGAS), Santiago de Compostela (ES); FUNDACIÓN PÚBLICA GALEGA INSTITUTO DE INVESTIGACIÓN SANITARIA DE SANTIAGO DE COMPOSTELA, Santiago de Compostela (ES); UNIVERSIDADE FEDERAL DE SERGIPE, Säo Cristóväo (BR)

(72) Inventors: Rodolfo GOMEZ BAHAMONDE, Santiago de Compostela (ES); James ALMADA DA SILVA, Säo Cristóväo (BR); Miriam LÓPEZ FAGÚNDEZ, Santiago de Compostela (ES); Eloi FRANCO TREPAT, Santiago de Compostela (ES); Ana ALONSO PÉREZ, Santiago de Compostela (ES); Maria GULLÁN FRESCO, Santiago de Compostela (ES); Andrés PAZOS PÉREZ, Santiago de Compostela (ES); Alberto Agustin JORGE MORA, Santiago de Compostela (ES)

(21) Appl. No.: 18/996,787

(22) PCT Filed: Jul. 20, 2023
(86) PCT No.: PCT/EP2023/070090

§ 371 Date: Jan. 17, 2025

(30) Foreign Application Priority Data
Jul. 20, 2022 (EP) 23382696.7

(51) Int. Cl. A61K 31/366

(52) U.S. Cl. CPC A61K 31/366

(57) ABSTRACT

The present invention refers to a composition for use in the prevention and/or treatment of diseases mediated by TLR4, IL1R, COX1/2 and/or RBP4, preferably inflammatory diseases such as Osteoarthritis (OA) or other diseases, such as infectious diseases, causing an inflammatory response.

### FIELD OF THE INVENTION

The present invention refers to the medical field. Particularly, the present invention refers to a composition for use in the prevention and/or treatment of diseases mediated by TLR4, IL1R, COX1/2 and/or RBP4, preferably inflammatory diseases such as, for example, Osteoarthritis (OA) or other diseases, such as infectious diseases, causing an inflammatory response.

### STATE OF THE ART

Inflammation is an important component of a wide variety of diseases. As important mediators of the inflammatory response, TLR4, IL1R, COX1/2 and RBP4 have been reported to be involved in the pathogenesis of multiple inflammatory diseases, such as OA.

OA is the most common chronic rheumatic disease. It is mainly characterized by joint space narrowing due to progressive degradation of the articular cartilage. These joint alterations are associated to pain, disability, and loss of joint architecture. Cartilage is the main tissue altered during OA progression. However, it also affects other joint tissues including bone, synovium, and tendons. Accordingly, it is considered a whole joint pathology. It is well-known that biomechanical alterations are the driving force in this pathology. Interestingly, it has been widely described that inflammation contributes to its onset and progression. This inflammation is frequently treated using cyclooxygenase enzyme (COX) inhibitors. Nonetheless, this pharmacological approach does not block OA-induced inflammation because OA inflammation is also sustained by innate immune response mediated innate immune receptors like TLR4 and IL1R.

It was demonstrated that COX enzymes have an active role in joint degenerative diseases. This enzyme regulates the formation of important biological mediators called prostanoids, including prostaglandins, prostacyclin's, and thromboxane's. In fact, prostaglandin (PG) causes pain during inflammation. An inflammatory environment induces COX-2 production and stimulates angiogenesis, supporting OA catabolic and inflammatory processes. According to COX properties, it was extensively used as a therapeutic target to treat inflammation and pain in OA and rheumatoid arthritis (RA) patients. However, it is well-known that COX inhibitors do not block the origin of OA inflammation and only help to control the symptoms, especially pain.

IL-1β belongs to a family of well-known pro-catabolic and inflammatory factors. It is an inducer of cartilage degradation but also drives synovitis. Accordingly, it has been demonstrated that IL-1β expression was higher in OA patients. Nonetheless, IL-1 blockade alone did not improve OA progression. However, IL-1 has proven to be a good target in other inflammatory rheumatic diseases like rheumatoid arthritis and gout, among others.

The receptors from the toll-like receptor family (TLRs) are also linked to the development of articular IIRs. TLRs can recognize pathogen-associated molecular patterns (PAMPs) but they also recognize damage-associated molecular patterns (DAMPS), which are host-derived molecules released by different cell types when tissues are damaged. These DAMPS and PAMPS are highly involved in the development of OA and other rheumatic diseases. TLRs, like TLR4, have been deeply involved in articular inflammatory responses in the OA context. However, currently, there is no approved inhibitor to block their activation.

Specifically, TLR4- and IL1R-mediated responses are associated with cytokine production like lipocalin-2 (LCN2) and interleukin-6 (IL-6), as well as to other catabolic and inflammatory-related factors such as nitric oxide synthase (NOS₂), cyclooxygenases enzymes (COX ½) and metalloproteinases (MMPs).

Multiple rheumatic diseases, including OA, are tightly linked with metabolic alterations that significantly contribute to their onset and progression. Among the factors supporting this relationship are adipokines, like RBP4. This adipokine has been associated with the development of several metabolic disorders such as diabetes, obesity, or metabolic syndrome. In addition, it also has a relevant role in other diseases characterised by a chronic inflammatory state like arthritis. Consistent with this, previous reports have revealed that RBP4 may bind to and activate Toll-like receptor 4 (TLR4). This means that RBP4 is considered a damage-associated molecular pattern (DAMP), which are factors released in pathologic circumstances where tissues are being damaged and participate in the inflammatory responses. Nonetheless, currently, there is no available therapy to reduce RBP4 activity.

Consequently, there is an unmet medical need of identifying new candidates to be used in the prevention and/or treatment of diseases mediated by TLR4, IL1R, COX1/2 and/or RBP4, preferably inflammatory diseases, such as OA, which, at the same time, do not show toxic effects.

The present invention is focused on solving this technical problem and new compounds for said intended medical use are herein provided.

### DESCRIPTION OF THE INVENTION

### Brief Description of the Invention

The present invention refers to a composition for use in the prevention and/or treatment of diseases mediated by TLR4, IL1R, COX1/2 and/or RBP4, preferably inflammatory diseases such as OA or other diseases, such as infectious diseases, causing an inflammatory response.

Particularly, three structurally related active compounds have been isolated and assayed in the present invention. Such as it is shown in the results provided in the present invention, in silico and in vitro experiments were performed in chondrocytes, synoviocytes and osteoblasts and the RT-PCR, biochemical assays, MTT assays, and western blot results revealed that these compounds exhibit anti-TLR4 (Example 2.2, 2.3, 2.4 and 2.5, anti-IL1R (Example 2.2, 2.3, 2.4 and 2.6), and anti-COX1/2 (Example 2.2 and 2.7) activities without showing any toxic effect. Moreover, these compounds are able to reduce the expression of RBP4 (Examples 2.8).

In agreement with these results, the compounds of the invention blocked TLR4-IL1R-mediated innate immune responses in primary human chondrocytes, in primary human osteoblasts and in human synoviocytes (Example 2.9.1, 2.9.2 and 2.9.3). Consequently, according to the data presented in the present invention, these compounds, through their anti-TLR4, anti-IL1R, and anti-COX1/2 activities, and the reduction of RBP4, might be a potential pharmacological tool in the prevention and/or treatment of diseases mediated by TLR4, IL1R, COX1/2 and/or RBP4, preferably inflammatory diseases such as OA.

It belongs to the common general knowledge that the diseases represented on Table 1, Table 2 and Table 3 are mediated by TLR4, IL1R and COX1/2, respectively. Inhibition of the respective molecular target has been reported to have a protective effect against these diseases, as indicated in the references provided in Table 1, Table 2 and Table 3. Thus, it is plausible to think that, since the compounds of the invention block TLR4, IL1R and COX1/2, they could be a pharmacological tool in the prevention and/or treatment of any of the diseases represented on Table 1, Table 2 and Table 3.

So, the first embodiment of the present invention refers to a compound of Formula (I) (herein after compound of the invention), or salts derived thereof, wherein OAc is an acetyloxy group and R may be substituted by OH, H or OAc,

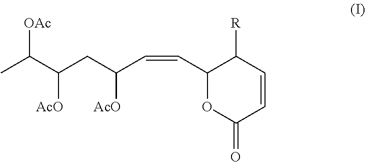

for use in the prevention and/or treatment of diseases mediated by TLR4, IL1R, COX1/2 and/or RBP4.

Kindly note that the compounds of the invention are also identified in the present invention as follows:
Pectinolide J (R=OH).
Hyptolide (R=H).
Pectinolide (R=OAc).

On the other hand, please note that the diseases which may be mediated by TLR4, IL1R, COX1/2 and/or RBP4 pertain to the common general knowledge. In other words, the person skilled in the art, using the common general knowledge, would undoubtedly recognize the diseases which are mediated by TLR4, IL1R, COX1/2 and/or RBP4. On the other hand, the Examples shown below comprise clear instructions allowing the person skilled in the art to recognise which conditions fall within the functional definition (i.e., diseases which are mediated by TLR4, IL1R, COX1/2 and/or RBP4).

For instance, according to the results provided in the present invention, the following inflammatory diseases, or other diseases, such as infectious diseases causing an inflammatory response, could be prevented and/or treated:
Diseases or conditions which may be treated through the blockade of TLR4 with the compounds of the present invention: Sepsis, SARS-COV infection, Human immunodeficiency virus (HIV) infection, Influenza A virus (IAV) infection, Respiratory syncytial virus (RSV) infection, Dengue (DENV) infection, Ebola virus (EBOV) infection, Chlamydia infection, Osteoarthritis (OA), Rheumatoid arthritis (RA), Ankylosing spondylitis, Gout, Heterotopic ossification, Fibrodysplasia ossificans progressive, Neuroinflammation, Traumatic brain injury (TBI), Systemic lupus erythematosus (SLE), Psoriasis/Psoriatic arthritis and/or IBD (inflammatory bowel disease).
Diseases or conditions which may be treated through the blockade of IL1R with the compounds of the present invention:
   Autoinflammatory periodic fever syndromes from two years of age:
      Cryopyrin-associated periodic syndromes (CAPS):
         Muckle-Wells syndrome.
         Neonatal Onset Multisystem Inflammatory Disease (NOMID).
         Chronic Infantile Neurological Cutaneous and Articular (CINCA) Syndrome.
         Familial Cold Autoinflammatory Syndrome (FCAS).
         Familial Cold Urticaria (FCU).
      Tumor necrosis factor receptor-associated periodic syndrome (TRAPS).
      Hyperimmunoglobulin D syndrome (HIDS).
      Mevalonate kinase deficiency (MKD).
      Familial Mediterranean Fever (FMF).
   Adult Still's disease and systemic juvenile idiopathic arthritis
   Gouty arthritis
   Rheumatoid Arthritis (RA)
   COVID-19
   Periodic febrile syndromes from 8 months of age:
      Cryopyrin-associated periodic syndromes (CAPS):
         Muckle-Wells syndrome
         Neonatal Onset Multisystem Inflammatory Disease (NOMID)
         Chronic Infantile Neurological Cutaneous and Articular (CINCA) Syndrome
         Familial Cold Autoinflammatory Syndrome (FCAS)
         Familial Cold Urticaria (FCU)
      Familial Mediterranean Fever (FMF)
   Adult Still's disease or systemic juvenile idiopathic arthritis
   Normal immune responses regulation
   Sepsis
   Human immunodeficiency virus (HIV)
   Osteoarthritis (OA)
   Ankylosing spondylitis
   Pseudogout
   Heterotopic ossification (HO)
Diseases or conditions which may be treated through the blockade of COX1/2 with the compounds of the present invention: Atopic dermatitis, Ankylosing spondylitis, Osteoarthritis (OA), Rheumatoid arthritis (RA), Gout, Heterotopic ossification (HO), Fibrodysplasia ossificans progressiva (FOP), Pain/Edema and/or Fever.
Diseases or conditions which may be treated through reduction of the expression of RBP4 with the compounds of the present invention: Insulin resistance, Inflammation, Retinopathies, Fatty liver and/or Hypertension.

In a preferred embodiment, the present invention refers to the compounds of the invention, or salts derived thereof, for use in the prevention and/or treatment of inflammatory diseases mediated by TLR4, IL1R, COX1/2 and/or RBP4, for example OA.

In a preferred embodiment, the compounds of the invention, or salts derived thereof, is administered topically, orally or by means of an intraarticular, subcutaneous, intra-venous or muscular injection.

The second embodiment of the present invention refers to a pharmaceutical composition (hereinafter pharmaceutical composition of the invention) comprising the compounds of the invention, or salts derived thereof, for use in the prevention and/or treatment of diseases mediated by TLR4, IL1R, COX1/2 and/or RBP4.

In a preferred embodiment, the present invention refers to the pharmaceutical composition of the invention for use in the prevention and/or treatment of inflammatory diseases mediated by TLR4, IL1R, COX1/2 and/or RBP4, for example OA.

In a preferred embodiment, the pharmaceutical composition of the invention is administered topically, orally or by means of an intraarticular, subcutaneous, intra-venous or muscular injection.

The third embodiment of the present invention refers to a method for the prevention and/or treatment of diseases mediated by TLR4, IL1R, COX1/2 and/or RBP4, preferably inflammatory diseases mediated by TLR4, IL1R, COX1/2 and/or RBP4, most preferably OA, which comprises administering to the patient a therapeutically effective dose or amount of the compound or pharmaceutical composition of the invention.

The compounds of the invention may be used alone or in combination with other therapies, either to complement their action or to reduce the concentration (and associated side effects) of a given drug.

On the other hand, as explained above, it is herein demonstrated that the compounds of the invention block COX1/2 activity (Example 2.2 and Example 2.7), thus making plausible the use of these compounds for the treatment of diseases mediated by COX1/2, preferably those represented on Table 1.

TABLE 1
Diseases mediated by COX1/2, and references indicating
that inhibition of that molecular target has a
protective effect against a given disease.

| Diseases mediated by COX1/2 | References |
|---|---|
| Osteoarthritis (OA) | Celecoxib/Indomethacin data |
| | sheet |
| Rheumatoid arthritis (RA) | Celecoxib/Indomethacin data |
| | sheet |
| Ankylosing spondylitis | Celecoxib/Indomethacin data |
| | sheet |
| Gout | Indomethacin data sheet |
| Pseudogout | Indomethacin data sheet |
| Heterotopic ossification (HO) | Prophylaxis treatment to prevent |
| | HO (1) |
| Inflammation/Pain/ | Indomethacin data sheet |
| Menstrual Pain/Trauma | |
| Atopic dermatitis | (2) |
| Fibrodysplasia ossificans | Prophylaxis treatment to prevent |
| progressive (FOP) | or ameliorates flare-ups in FOP. |
| | FOP Treatment Guidelines (pdf.) |
| | (3)(4)(5)(6)(7) |
| Fever | Aspirin Data Sheet |

Celecoxib is a nonsteroidal anti-inflammatory drug (NSAID) that selectively inhibits the cyclooxygenase-2 (COX-2) enzyme. Celecoxib is used to treat pain, inflammation, and stiffness caused by conditions such as osteoarthritis, rheumatoid arthritis, and menstrual pain.

Celecoxib is primarily used to treat pain and inflammation associated with certain conditions, such as:
Osteoarthritis: a type of arthritis caused by the wear and tear of cartilage in the joints.
Rheumatoid arthritis: an autoimmune disorder that causes inflammation in the joints and other parts of the body.
Ankylosing spondylitis: a type of arthritis that affects the spine.
Juvenile rheumatoid arthritis: a form of arthritis that affects children.
Menstrual pain: pain and cramps associated with the menstrual cycle.

Indomethacin is a nonsteroidal anti-inflammatory drug (NSAID) that is a non-selective cyclooxygenase (COX) inhibitor, meaning that it blocks both the COX-1 and COX-2 enzymes. COX enzymes play a role in the production of prostaglandins, which are substances that contribute to inflammation and pain. By inhibiting these enzymes, indomethacin reduces inflammation and pain associated with certain conditions, such as:
Rheumatoid arthritis: an autoimmune disorder that causes inflammation in the joints and other parts of the body.
Osteoarthritis: a type of arthritis caused by the wear and tear of cartilage in the joints.
Ankylosing spondylitis: a type of arthritis that affects the spine.
Gout: a type of arthritis caused by the build-up of uric acid crystals in the joints.
Heterotopic ossification: Currently indomethacin acts as a prophylaxis treatment to prevent HO.

The compounds of the invention block IL1R activity (Example 2.2, 2.3, 2.4, 2.6, 2.9.1, 2.9.2 and 2.9.3), thus making plausible the use of these compounds for the treatment of diseases mediated by IL1R, preferably those represented on Table 2.

TABLE 2
Diseases mediated by IL1R response, and references
indicating that inhibition of that molecular target
has a protective effect against a given disease.

| Diseases mediated by IL1R | References |
|---|---|
| Tumor necrosis factor receptor-associated | (8) |
| periodic syndrome (TRAPS) | |
| Hyperimmunoglobulin D syndrome (HIDS) | (3) (4) |
| Mevalonate kinase deficiency (MKD) | (5)(2) |
| Human immunodeficiency virus (HIV) | (6)(7)(14) |
| Sepsis | (15) |
| Familial Mediterranean Fever (FMF) | Kineret/Anakinra |
| | data sheet |
| Adult Still's disease and systemic | Kineret/Anakinra |
| juvenile idiopathic arthritis | data sheet |
| COVID-19 | Kineret/Anakinra |
| | data sheet |
| Cryopyrin-associated periodic syndromes (CAPS): | Kineret/Anakinra |
| Muckle-Wells syndrome | data sheet |
| Neonatal Onset Multisystem Inflammatory Disease | |
| (NOMID) | |
| Chronic infantile neurological cutaneous and | |
| articular (CINCA) syndrome | |
| Familial Cold Autoinflammatory Syndrome | |
| (FCAS) | |
| Familial Cold Urticaria (FCU) | |
| Rheumatoid arthritis (RA) | Kineret/Anakinra |
| | datasheet |
| Gouty arthritis | (16)(17) |
| Osteoarthritis (OA) | (17)(18)(19) |
| Ankylosing spondylitis | (17)(20) |
| Pseudogout | (17) |
| Heterotopic ossification (HO) | (21)(22) |

IL-1 targeting has been reported as a useful treatment for all the diseases represented on Table 2. In support with this, Anakinra, a well-known IL-1 receptor antagonist, has been approved to treat several of these diseases.

The compounds of the invention block TLR4 activity (Example 2.2, Example 2.3, Example 2.4, Example 2.5, Example 2.9.1 and Example 2.9.2, 2.9.3), thus making plausible the use of these compounds for the treatment of diseases mediated by TLR4, preferably those represented on Table 3.

TABLE 3
Diseases mediated by TLR4 response, and references
indicating that inhibition of that molecular target
has a protective effect against a given disease.

| Diseases mediated by TLR4 | References |
|---|---|
| Osteoarthritis (OA) | (23)(24)(25)(26) |
| Rheumatoid arthritis (RA) | (24)(26) |
| Gout | (27)(28)(29) |
| Ankylosing spondylitis | (26) |
| Heterotopic ossification (HO) | (30)(31) |
| Fibrodysplasia ossificans progressive (FOP) | (32) |
| Psoriasis/Psoriatic arthritis | (33)(34) |
| and/or IBD (inflammatory bowel disease) | |
| Systemic lupus erythematosus (SLE) | (35)(36)(37) |
| Traumatic brain injury (TBI) | (38)(39)(40) |
| Neuroinflammation | (41) (42) (43) |
| SARS-CoV infection | (44)(45) |
| Sepsis | (46) |
| Human immunodeficiency virus (HIV) | (47) |
| Influenza A virus (IAV) | |
| Respiratory syncytial virus (RSV) | |
| Dengue (DENV) | |
| Ebola virus (EBOV) | |
| Chlamydia infection | |
| Reactive arthritis (ReA) | (48) (49) |
| Septic arthritis (SeA) | (50) (51) |

The compounds of the invention block RBP4 activity (Example 2.8), thus making plausible the use of these compounds for the treatment of diseases mediated by RBP4, preferably those represented on Table 4.

TABLE 4
Diseases mediated by RBP4, and references indicating
that inhibition of that molecular target has a
protective effect against a given disease.

| Diseases mediated by RBP4 | References |
|---|---|
| Insulin resistance | (52) (53) (54) |
| Retinopathy | (55) (56) (57) (58) |
| Fatty liver disease | (59) (60) |
| Hypertension | (59) (61) |

Consequently, in addition to the aspects described above, the present invention also refers to the compound of Formula (I), or salts derived thereof, wherein OAc is an acetyloxy group and R is OH, H or OAc,

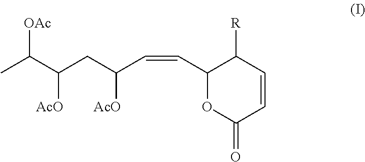

for use in the prevention and/or treatment of a disease selected from the group comprising: osteoarthritis, rheumatoid arthritis, ankylosing spondylitis, gout, pseudogout, heterotopic ossification, menstrual pain, atopic dermatitis, fibrodysplasia ossificans progressive (FOP), fever, tumour necrosis factor receptor-associated periodic syndrome (TRAPS), hyperimmunoglobulin D syndrome (HIDS), mevalonate kinase deficiency (MKD), human immunodeficiency virus (HIV) infection, sepsis, familial Mediterranean fever (FMF), septic arthritis, reactive arthritis, adult Still's disease, systemic juvenile idiopathic arthritis, COVID-19, cryopyrin-associated periodic syndromes (CAPS), gouty arthritis, psoriasis, psoriatic arthritis, inflammatory bowel disease (IBD), systemic lupus erythematosus (SLE), traumatic brain injury (TBI), neuroinflammation, influenza A virus (IAV) infection, respiratory syncytial virus (RSV) infection, dengue infection, Ebola virus disease or Chlamydia infection, insulin resistance, retinopathy, fatty liver disease or hypertension.

In a preferred embodiment, the compound is for use in the prevention and/or treatment of a disease mediated by the activity of TLR4, IL1R, COX1/2 and/or RBP4 in chondrocytes, human osteoblasts and/or in synoviocytes

In a preferred embodiment the disease is mediated by:
a) COX1/2, and it is selected from the group comprising: osteoarthritis, rheumatoid arthritis, ankylosing spondylitis, gout, pseudogout, heterotopic ossification, menstrual pain, atopic dermatitis, fibrodysplasia ossificans progressive (FOP) or fever;
b) IL1R, and it is selected from the group comprising: tumor necrosis factor receptor-associated periodic syndrome (TRAPS), hyperimmunoglobulin D syndrome (HIDS), mevalonate kinase deficiency (MKD), human immunodeficiency virus (HIV) infection, sepsis, familial Mediterranean fever (FMF), adult Still's disease, systemic juvenile idiopathic arthritis, COVID-19, Muckel-Wells syndrome, neonatal onset multisystem inflammatory disease (NOMID), chronic infantile neurological cutaneous and articular (CINCA) syndrome, familial cold autoinflammatory syndrome (FCAS), familial cold urticaria (FCU), rheumatoid arthritis, gouty arthritis, osteoarthritis, ankylosing spondylitis, pseudogout or heterotopic ossification;
c) TLR4, and it is selected from the group comprising: osteoarthritis, rheumatoid arthritis, gout, ankylosing spondylitis, heterotopic ossification, fibrodysplasia ossificans progressive (FOP), psoriasis, psoriatic arthritis, inflammatory bowel disease (IBD), systemic lupus erythematosus (SLE), traumatic brain injury (TBI), neuroinflammation, SARS-COV infection, sepsis, human immunodeficiency virus (HIV) infection, influenza A virus (IAV) infection, respiratory syncytial virus (RSV) infection, dengue infection, Ebola virus disease or Chlamydia infection. In addition, TLR4 responses play a role in the development of reactive arthritis (ReA), which is a condition that occurs following an infection with diverse gram negative bacteria such as Chlamydia (48) (49). Furthermore, it has been well-documented that bacterial joint infection or the presence of sepsis contributes to the development of septic arthritis. Therefore, septic arthritis was also chosen as a potential target (50) (51).
d) RBP4, and it is selected from the group comprising: insulin resistance, retinopathy, fatty liver disease or hypertension.

In a preferred embodiment the compound is for use in the prevention and/or treatment of diseases mediated by the activity of TLR4, IL1R and/or COX1/2, preferably involving osteoblasts, chondrocytes or synoviocytes, selected from the group comprising: osteoarthritis, rheumatoid arthritis, ankylosing spondylitis, heterotopic ossification, adult Still's disease, systemic juvenile idiopathic arthritis, cryopyrin-associated periodic syndromes (CAPS), gouty arthritis, pseudogout, tumour necrosis factor receptor-associated periodic syndrome (TRAPS), familial Mediterranean fever (FMF), septic arthritis, reactive arthritis, fibrodysplasia ossificans progressive (FOP), psoriatic arthritis or systemic lupus erythematosus (SLE).

In a preferred embodiment, the cryopyrin-associated periodic syndrome (CAPS) is selected from the group comprising: Muckel-Wells syndrome, neonatal onset multisystem inflammatory disease (NOMID), chronic infantile neurological cutaneous and articular (CINCA) syndrome, familial cold autoinflammatory syndrome (FCAS) or familial cold urticaria (FCU).

In a preferred embodiment, the compound is for use in the prevention and/or treatment of osteoarthritis.

In a preferred embodiment, the compound is administered topically, orally or by means of an intraarticular, subcutaneous, intravenous or muscular injection.

Alternatively, the present invention also refers to a pharmaceutical composition comprising a compound of Formula (I), or salts derived thereof, wherein OAc is an acetyloxy group and R is OH, H or OAc, and, optionally, excipients or carriers pharmaceutically acceptable, for use in the prevention and/or treatment of a disease selected from the group comprising: osteoarthritis, rheumatoid arthritis, ankylosing spondylitis, gout, pseudogout, heterotopic ossification, menstrual pain, atopic dermatitis, fibrodysplasia ossificans progressive (FOP), fever, tumour necrosis factor receptor-associated periodic syndrome (TRAPS), hyperimmunoglobulin D syndrome (HIDS), mevalonate kinase deficiency (MKD), human immunodeficiency virus (HIV) infection, sepsis, familial Mediterranean fever (FMF), septic arthritis, reactive arthritis, adult Still's disease, systemic juvenile idiopathic arthritis, COVID-19, cryopyrin-associated periodic syndromes (CAPS), gouty arthritis, psoriasis, psoriatic arthritis, inflammatory bowel disease (IBD), systemic lupus erythematosus (SLE), traumatic brain injury (TBI), neuroinflammation, influenza A virus (IAV) infection, respiratory syncytial virus (RSV) infection, dengue infection, Ebola virus disease or Chlamydia infection, insulin resistance, retinopathy, fatty liver disease or hypertension.

In a preferred embodiment, the pharmaceutical composition is for use in the prevention and/or treatment of a disease mediated by the activity of TLR4, IL1R, COX1/2 and/or RBP4 in chondrocytes, human osteoblasts and/or in synoviocytes.

In a preferred embodiment the disease is mediated by:
a) COX1/2, and it is selected from the group comprising: osteoarthritis, rheumatoid arthritis, ankylosing spondylitis, gout, pseudogout, heterotopic ossification, menstrual pain, atopic dermatitis, fibrodysplasia ossificans progressive (FOP) or fever;
b) IL1R, and it is selected from the group comprising: tumor necrosis factor receptor-associated periodic syndrome (TRAPS), hyperimmunoglobulin D syndrome (HIDS), mevalonate kinase deficiency (MKD), human immunodeficiency virus (HIV) infection, sepsis, familial Mediterranean fever (FMF), adult Still's disease, systemic juvenile idiopathic arthritis, COVID-19, Muckel-Wells syndrome, neonatal onset multisystem inflammatory disease (NOMID), chronic infantile neurological cutaneous and articular (CINCA) syndrome, familial cold autoinflammatory syndrome (FCAS), familial cold urticaria (FCU), rheumatoid arthritis, gouty arthritis, osteoarthritis, ankylosing spondylitis, pseudogout or heterotopic ossification;
e) TLR4, and it is selected from the group comprising: osteoarthritis, rheumatoid arthritis, gout, ankylosing spondylitis, heterotopic ossification, fibrodysplasia ossificans progressive (FOP), psoriasis, psoriatic arthritis, inflammatory bowel disease (IBD), systemic lupus erythematosus (SLE), traumatic brain injury (TBI), neuroinflammation, SARS-COV infection, sepsis, human immunodeficiency virus (HIV) infection, influenza A virus (IAV) infection, respiratory syncytial virus (RSV) infection, dengue infection, Ebola virus disease or Chlamydia infection. In addition, TLR4 responses play a role in the development of reactive arthritis (ReA), which is a condition that occurs following an infection with diverse gram negative bacteria such as Chlamydia (48) (49). Furthermore, it has been well-documented that bacterial joint infection or the presence of sepsis contributes to the development of septic arthritis. Therefore, septic arthritis was also chosen as a potential target (50) (51).
c) RBP4, and it is selected from the group comprising: insulin resistance, retinopathy, fatty liver disease or hypertension.

In a preferred embodiment the compound is for use in the prevention and/or treatment of diseases mediated by the activity of TLR4, IL1R and/or COX1/2, preferably involving osteoblasts, chondrocytes or synoviocytes, selected from the group comprising: osteoarthritis, rheumatoid arthritis, ankylosing spondylitis, heterotopic ossification, adult Still's disease, systemic juvenile idiopathic arthritis, cryopyrin-associated periodic syndromes (CAPS), gouty arthritis, pseudogout, tumour necrosis factor receptor-associated periodic syndrome (TRAPS), familial Mediterranean fever (FMF), septic arthritis, reactive arthritis, fibrodysplasia ossificans progressive (FOP), psoriatic arthritis or systemic lupus erythematosus (SLE).

In a preferred embodiment, the cryopyrin-associated periodic syndrome (CAPS) is selected from the group comprising: Muckel-Wells syndrome, neonatal onset multisystem inflammatory disease (NOMID), chronic infantile neurological cutaneous and articular (CINCA) syndrome, familial cold autoinflammatory syndrome (FCAS) or familial cold urticaria (FCU).

In a preferred embodiment, the pharmaceutical composition is for use in the prevention and/or treatment of osteoarthritis.

In a preferred embodiment, the pharmaceutical composition is administered topically, orally or by means of an intraarticular, subcutaneous, intravenous or muscular injection.

For the purpose of the present invention the following terms are defined:
The term “comprising” is meant including, but not limited to, whatever follows the word “comprising”. Thus, use of the term “comprising” indicates that the listed elements are required or mandatory, but that other elements are optional and may or may not be present.
By “consisting of” is meant including, and limited to, whatever follows the phrase “consisting of”. Thus, the phrase “consisting of” indicates that the listed elements are required or mandatory, and that no other elements may be present.
By “therapeutically effective dose or amount” is intended an amount that, when administered as described herein, brings about a positive therapeutic response in the patient. The exact amount required will vary from subject to subject, depending on (non-exhaustive list): the species, age, general condition of the subject, the severity of the condition being treated or the mode of administration. An appropriate “effective” amount in any individual case may be determined by one of ordinary skill in the art using routine experimentation, based upon the information provided herein.

### BRIEF DESCRIPTION OF THE FIGURES

FIG. 1. (A) Chemical structures of the compounds of the invention: Pectinolide J (R=OH). Hyptolide (R=H). Pectinolide E (R=OAc). (B) In silico conformational diversity analysis of crystallographic structures. Binding affinity of the compounds of the invention against the key inflammatory targets (TLR4, IL1R, COX1 and COX2). (C—H) Effect of the compounds of the invention on cell viability ATDC5 cells (MTT assay). Values are the mean and SEM of at least 3 independent experiments. The Fetal bovine serum (FBS) acts as a positive control. Statistical significance has been determined by One-Way ANOVA. (I-K) The compounds of the invention suppress lipopolysaccharide (LPS)-induced or (IL1B)-induced nitric oxide (NO) production. ATDC5 cells (1×10⁵) were stimulated with LPS (100 ng/ml) or IL1β mouse (0.5 ng/ml) and co-stimulated with several doses of Hyp (I), PE (J) and PJ (K) for 24 hours. NO concentration was determined using Griess reaction. Values are the mean and SEM of at least 3 independent experiments. Statistical significance has been determined by One-Way ANOVA.

FIG. 2. Effect of the compounds of the invention on pro-inflammatory TLR4-induced mRNA expression. ATDC5 cells (1×10⁵) were stimulated with LPS (100 ng/ml) and co-stimulated with several doses of the compounds of the invention. The mRNA expression of (A, D, G) lipocalin-2 (LCN2), (B, E, H) interleukin-6 (IL-6) and (C, F, I) nitric oxide synthase 2 (NOS2) were determined by performing real-time PCR (RT-PCR). Values are the mean and SEM of at least 3 independent experiments. Statistical significance has been determined by One-Way ANOVA.

FIG. 3. Effect of the compounds of the invention on pro-inflammatory IL1-induced mRNA expression. ATDC5 cells (1×10⁵) were stimulated with IL1B (0.5 ng/ml) and co-stimulated with several doses of the compounds of the invention. The mRNA expression of (A, D, G) lipocalin-2 (LCN2), (B, E, H) interleukin-6 (IL-6) and (C, F, I) nitric oxide synthase 2 (NOS2) were determined by performing real-time PCR (RT-PCR). Values are the mean and SEM of at least 3 independent experiments. Statistical significance has been determined by One-Way ANOVA.

FIG. 4. Effect of the compounds of the invention on pro-inflammatory TLR4-IL1-induced protein expression. (A-F) Blots were performed with protein lysates from ATDC5 cells treated for 24 h with the compounds of the invention and LPS (100 ng/ml) or IL1B (0.5 ng/ml) to detect NOS2 and b-actin (an internal standard). Values are the mean and SEM of at least 3 independent experiments. Statistical significance has been determined by One-Way ANOVA.

FIG. 5. Effects of the compounds of the invention on cyclooxygenase activity (A) (B). Indomethacin did not suppress lipopolysaccharide (LPS)-induced or (IL1β)-induced nitric oxide (NO) production (C). Statistical significance has been determined by One-Way ANOVA.

FIG. 6. Inhibition of RBP4 expression by PJ (50 and 100 μM of PJ) in the presence or absence of inflammatory stimuli (LPS and IL1β). (A-B) Values are the mean and SEM of at least 3 independent experiments. Statistical significance has been determined by One-Way ANOVA.

FIG. 7. The compounds of the invention blocked TLR4- and IL1-mediated innate immune responses in primary human chondrocytes cells. Primary chondrocytes cells (250.000 cells/well) were stimulated with (A-I) LPS (100 ng/ml) or (J-R) IL1β (0.5 ng/ml) and co-stimulated with 50 μM of the compounds of the invention. Values are the mean and SEM of at least 3 independent experiments. Statistical significance has been determined by One-Way ANOVA followed by a pertinent post-test. Comment: FIGS. 6A-E and G-I were already included in the EP priority application.

FIG. 8. The compounds of the invention blocked TLR4- and IL1-mediated innate immune responses in primary human osteoarthritis osteoblasts. Primary osteoblasts cells (100000 cells/well) were stimulated with (A, B, E, F, I, J) LPS (100 ng/ml) or (C, D, G, H, K, L) IL1β (0.5 ng/ml) and co-stimulated with 50 μM of the compounds of the invention. The pro-inflammatory and catabolic mRNA expression was determined by performing real-time PCR (RT-PCR). Values are the mean and SEM of at least 3 independent experiments. Statistical significance has been determined by One-Way ANOVA followed by a pertinent post-test.

FIG. 9. The compounds of the invention blocked TLR4-mediated innate immune responses in human synoviocytes. Synoviocytes cells (SW982) (100.000 cells/well) were stimulated with (A-C) LPS (100 ng/ml) or (D-F) IL1β (0.5 ng/ml) and co-stimulated with with 50 μM of Pectinolide J compound (as a representative compound of the compounds of the invention). The pro-inflammatory and catabolic mRNA expression (LCN2, IL6, CCL2) was determined by performing real-time PCR (RT-PCR). Values are the mean and SEM of at least 3 independent experiments. Statistical significance has been determined by One-Way ANOVA followed by a pertinent post-test.

### DETAILED DESCRIPTION OF THE INVENTION

The present invention is illustrated by means of the Examples set below without the intention of limiting its scope of protection.

### Example 1. Materials and Methods

### Example 1.1 General Experimental Procedures

Column chromatography was performed on silica gel (70-230 mesh, Macherey-Nagel) and fractions were monitored by TLC on silica gel plates (GF254, Merck), using the vanillin-sulfuric acid as a visualization reagent. HPLC (Shimadzu, Prominence model) was performed using LC-6AD pumps coupled with analytical or semi-preparative Waters C18 columns (250×4.6 and 250×21.2 mm, respectively). The HPLC system employed an SPD-20A UV-Vis detector. 1H and 13C NMR spectra were obtained using a Bruker AVANCE-III 11.74 Tesla (499.87 MHz for 1H and 125 MHz for 13C) in CD3OH solvent and SiMe4 as an internal standard. MS data were recorded on a Waters Xevo QTof MS. Absorbance data and optic rotations were determined in Cary 50 UV-Vis spectrophotometer and Perkin Elmer model 341 Polarimeter, respectively. All solvents used are of commercial HPLC grade.

### Example 1.2. Plant Material

Hyptis pectinata (L.) Poit was collected in September 2015. For the extraction process, the inflorescences were dried in an oven with air renewal and circulation at 40° C. until complete dehydration (4 days). Then, the vegetal material was triturated to yield a fine powder.

### Example 1.3. Extraction and Isolation of the Compounds of the Invention

The powdered inflorescence (0.88 kg) was extracted with ethanol (3×4.4 L) at room temperature (25° C.) during 10 minutes for each cycle in high-performance disperser (TE-102 Turratec®, Tecnal, Brazil) to 21,000 rpm. After filtration, the solvent was evaporated under reduced pressure in a rotary vacuum evaporator at 40° C. The resulting extract (62.53 g) was dissolved in EtOH—H2O mixture (6:4, v/v) and extracted with CH₂Cl₂ (3×200 mL). The CH₂Cl₂ layer was passed through the Na2SO4 and evaporated under reduced pressure at 40° C., which afforded a dark-coloured gum (HPFID, 57.84 g). Then, 20.00 g of gum was chromatographed on a silica gel (70-230 mesh) column (47 cm×6 cm i.d.) with a gradient system of hexane-CH₂Cl₂-Me2CO (6:2:2, 500 mL; 4:4:2, 650 mL; 4:2:4, 2650 mL) to yield seventy-six sub-fractions (50 mL each). Some fractions were combined after monitoring by TLC resulting in eight sub-fractions (HPFID-1, HPFID-2, HPFID-3, HPFID-4, HPFID-5, HPFID-6, HPFID-7, HPFID-8). HPFID-4 (1.2 g) was performed by HPLC analytical scale using reverse phase column (250×4.6 mm, 10μ) at 25° C., mobile phase MeOH—H2O (50:50), detection wavelength at 210 nm and the flow rate at 1.0 mL/min. Then, the compounds 1 (65.7 mg), 2 (23.9 mg) and 3 (123.9 mg) were isolated by semipreparative HPLC on a Waters column (250×21.2 mm, 10) at 25° C., and detected at 210 nm at a flow rate of 16.0 mL/min using a mobile phase MeOH—H₂O (50:50). The retention time for 1, 2 and 3 were 13.3, 20.6 and 27.0 min, respectively.

### Example 1.4. Cell Culture

ATDC5 murine chondrogenic cell line was obtained from RIKEN Cell Bank and cultured in DMEM/Ham's F-12 medium supplemented with 5% FBS, 2% glutamine, and 50 U/mL of penicillin and 50 μg/ml of streptomycin, 10 μg/mL human transferrin and 3× 10-8 M sodium selenite.

Otherwise, human synovial sarcoma SW982 (ATCC Cat #HTB-93, RRID: CVCL_1734) was performed in P12 cell culture plates (10⁵ cells per well). SW982 and primary cells were cultured in DMEM/Ham's F-12 medium supplemented with 10% FBS, 2% glutamine, and 50 U/mL of penicillin and 50 μg/ml of streptomycin.

All patients signed an informed con-sent approved by the Santiago Hospital Ethics Committee (CAEIG-2016/258). Primary human chondrocytes were isolated from cartilage matrix using pronase and collagenase digestion (Merk Sigma-Aldrich, Germany), whereas primary human osteoblasts were obtained from smiting the trabecular bone into flat pieces (Villalvilla et al., 2014).

### Example 1.5. Reagents

Foetal bovine serum (FBS), DMEM F-12 medium, antibiotics, trypsin, 1-glutamine, sodium selenite, methyl-thiazolyl-tetrazolium (MTT), transferrin, LPS (E. coli O127:B8) and primers (HPRT, LCN2, IL6, NOS2, and MMP3) were purchased from Sigma-Aldrich (St. Louis, MO, USA). IL-1β was purchased from Peprotech (NJ, USA).

DNAase I, buffer 10× was purchased from Lucigen (A Coruña, Spain). Retro transcription kit (RT) and ultrapure water were purchased from Applied Biosystems Thermo Fisher (Waltham, MA, USA). To do PCRs, master mix was purchased from BioRad (Alcobendas, Madrid, Spain).

Primary antibodies (iNOS and b-actin) were purchased from Applied Biosystems Thermo Fisher (Waltham, MA, USA). Secondary antibodies (IG mouse and IG rabbit) were purchased from Jackson Laboratories (Jackson ImmunoResearch Laboratories Inc., USA).

### Example 1.6. Molecular Docking Simulations

The compound structural data were obtained from a Protein Data Bank. Molecular docking simulations were conducted using Auto Dock Vina Software and the ensemble docking method. This software led to study the binding affinity between ligand (receptor) and protein (the compounds of the invention). This software showed the predicted and experimental free energies of binding for the data set (kcal/mol). A threshold criterion (docking score>−5.0 kcal/mol) was set to select the strongest interactions.

### Example 1.7. Cell Treatments

ATDC-5 cells were seeded in 12-well plates. Cells were seeded in a monolayer. After adhesion and incubation overnight in serum-free conditions (starvation), ATDC5 cells were stimulated for 24 h with LPS [100 ng/ml] or IL-1B [0.5 ng/ml] in the presence or absence of the compounds of the invention [10-100 μM].

### Example 1.8. Cell Viability

Viability was tested using the MTT reagent as previously described. Briefly, 8×10³ cells/well were plated in 96-well plates and treated as described above. Then, the cells were incubated for 4 h with MTT reagent. After formazan salt was dissolved, absorbance was measured at 550 nm in a microplate reader.

### Example 1.9. Nitrite Assay

As described above, 1×10⁵ ATDC5 cells were seeded and treated for 24 h. Nitrite accumulation was determined in the culture medium using the Griess reaction. As previously described, after diazonium salt was dissolved, absorbance was measured at 550 nm in a microplate reader.

### Example 1.10. Cyclooxygenase (COX) Activity Assay

The anti-inflammatory activity of the compounds of the invention was tested in terms of COX inhibition. The COX inhibition effect of the compounds of the invention was estimated using a Fluorometric Cyclooxygenase COX activity assay (Abcam, ab204699) according to the manufacturer's protocol.

After the addition of arachidonic acid (COX substrate), the enzymatic reaction led to the production of a fluorescent molecule (resorufin dye 2=535 nm), that could be monitored in kinetic mode (4 reads per min) for 60 min at room temperature (Epoch 2 de Bio Tek).

### Example 1.11. RNA Extraction and RT-PCR

RNA was extracted using E.Z.N.A solation reagent (Omega) according to manufacturer's protocol. Relative quantitation was performed using the ΔΔC Comparative Method and the MxPro software (Stratagene, CA, USA) as the ratio of each gene to the expression of the housekeeping gene. Data are shown as mean±S.E.M (error bars) of at least three independent experiments and represented as fold-change vs. controls.

### Example 1.12. Protein Extraction and Western Blotting

Whole-cell protein extraction was performed using lysis buffer (RIPA Buffer). Immunoblots were incubated with the appropriated antibodies [INOS (rabbit polyclonal antibody) diluted 1:1000 (Thermo Fisher Scientific, MA, USA), IG (anti-rabbit antibody) diluted 1:5000 (Jackson ImmunoResearch Laboratories Inc., USA), b-actin (mouse polyclonal antibody) (Thermo Fisher Scientific, MA, USA) diluted 1:6500 and IG (anti-mouse antibody) diluted (Jackson ImmunoResearch Laboratories Inc., USA).

Then, all immunoblots were visualized using an Immobilon Western kit (Millipore, MA, USA). Blots densitometries were generated using a ChemiDoc MP Imaging System and analysed using Image J software (National Institutes of Health, Bethesda, MD, USA).

### Example 1.12. Statistical Analysis

Data were reported as mean±SEM for at least three independent experiments. Statistical analyses were performed by analysis of variance followed by the One-Way Anova multiple comparison test or t-test analysis using Prism statistical analysis (GraphPad Software). p<0.05 was considered significant.

### Example 2. Results

### Example 2.1. Isolation and Characterization of the Compounds of the Invention

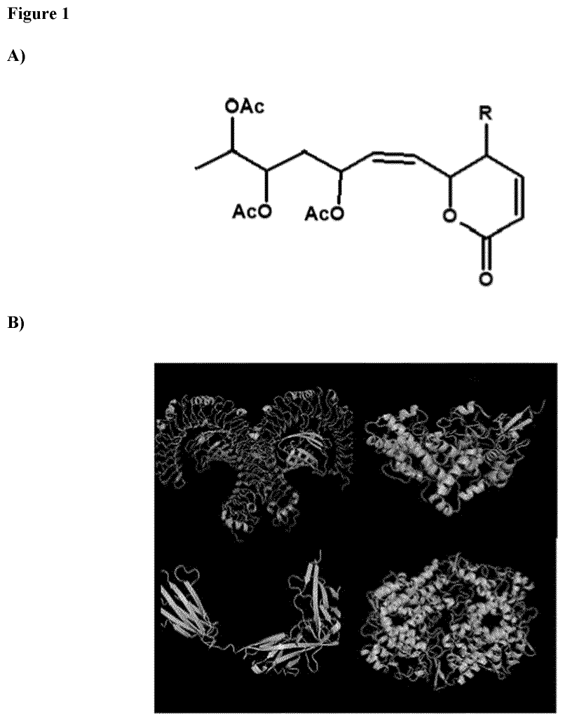

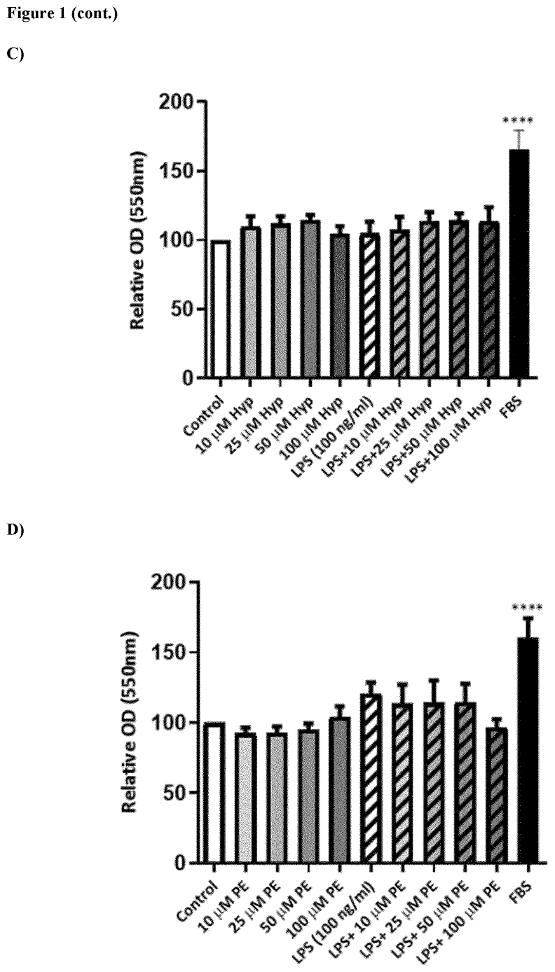

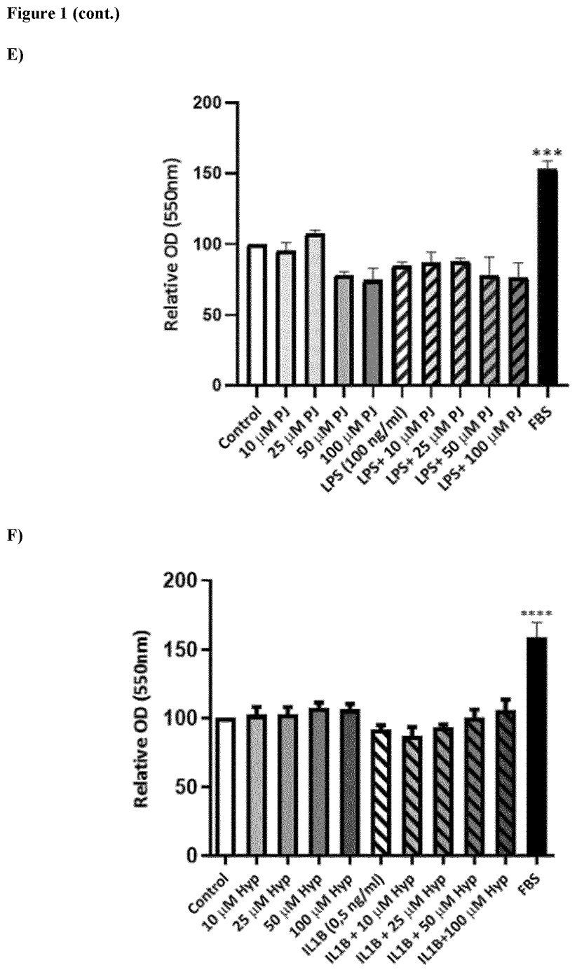

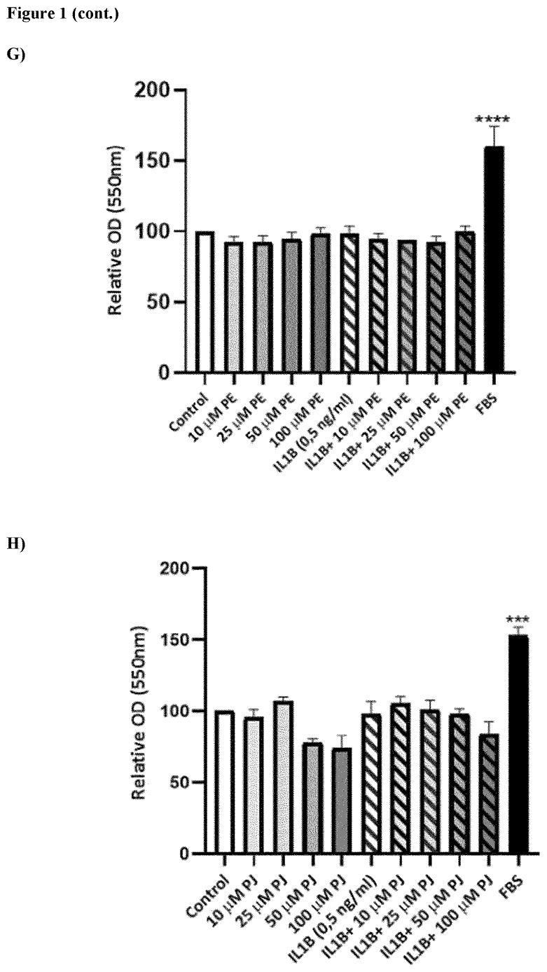

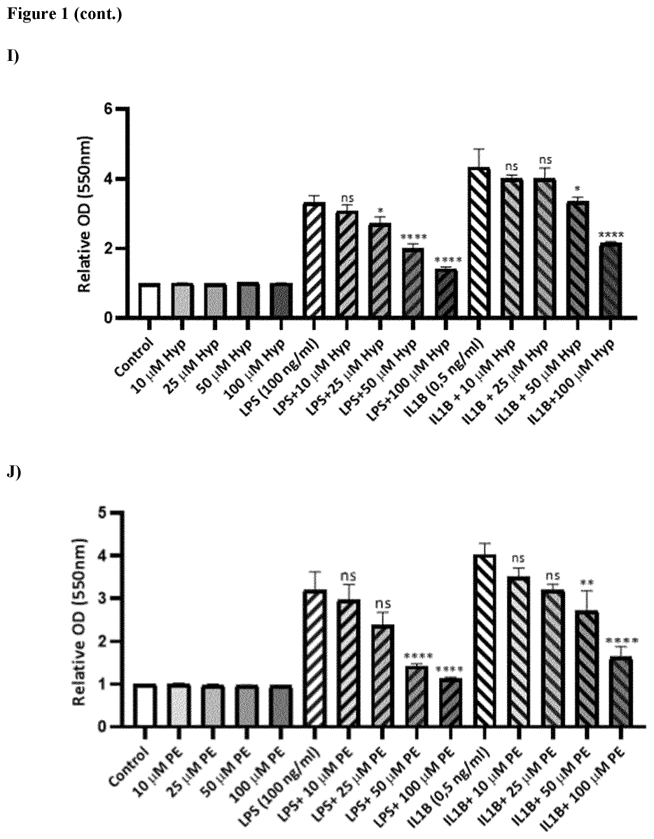

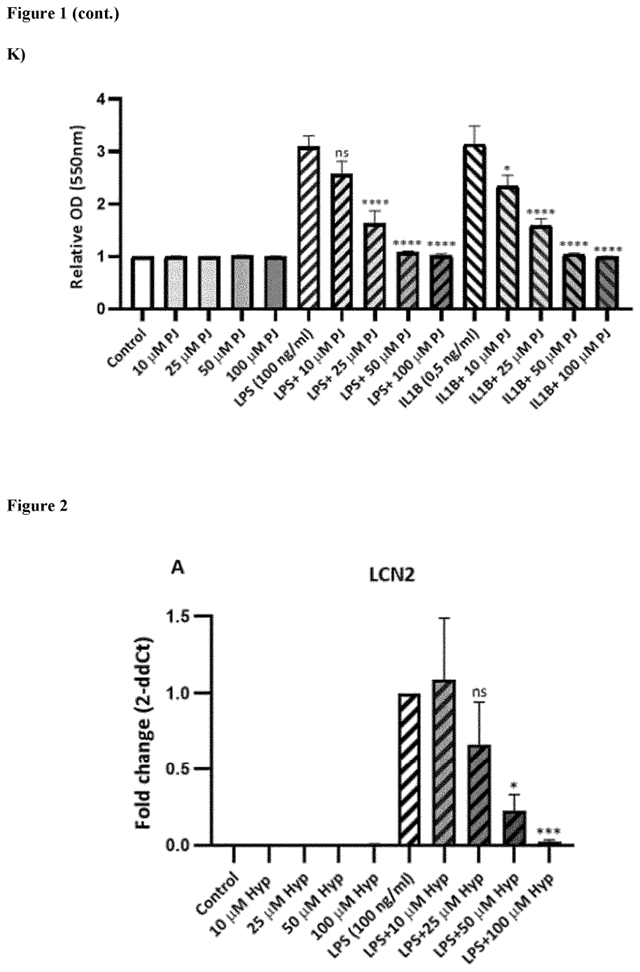

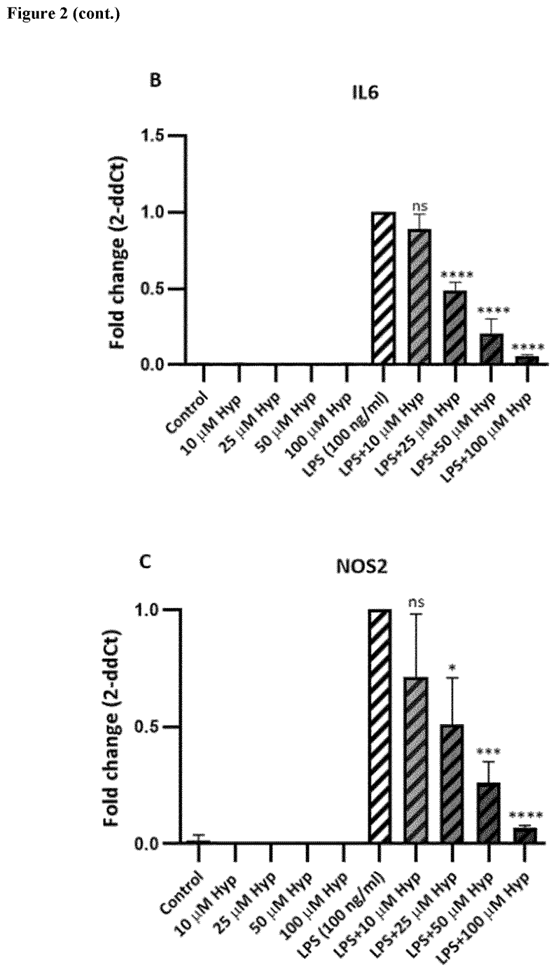

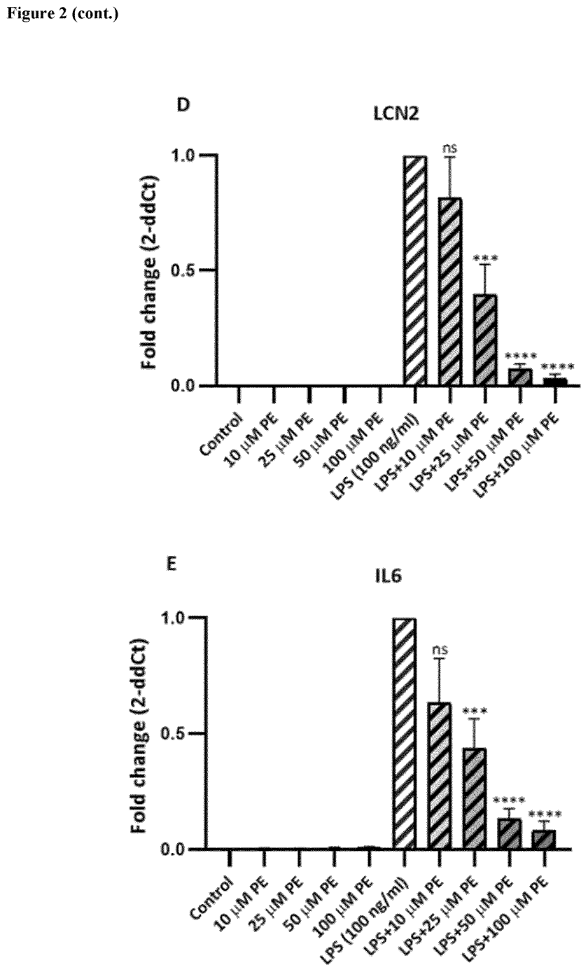

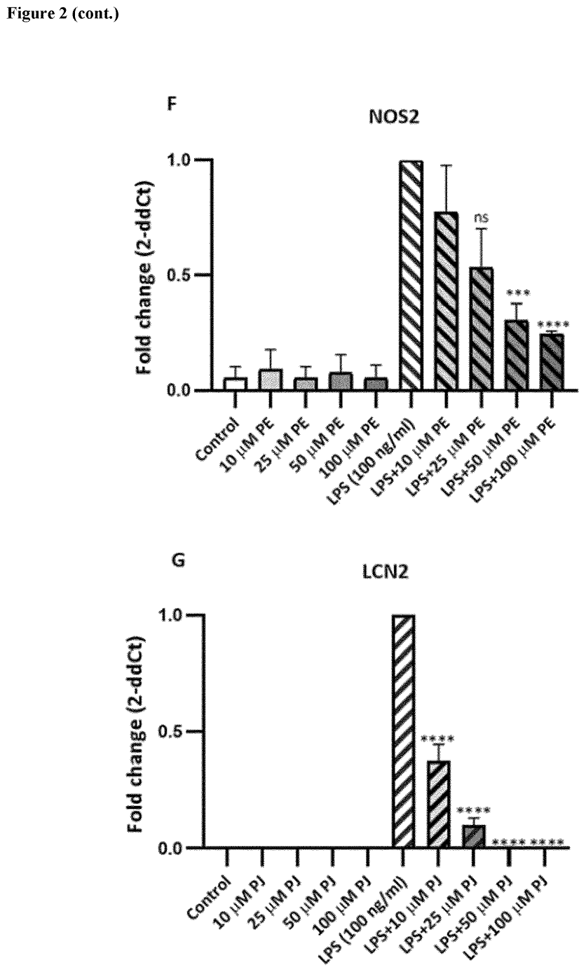

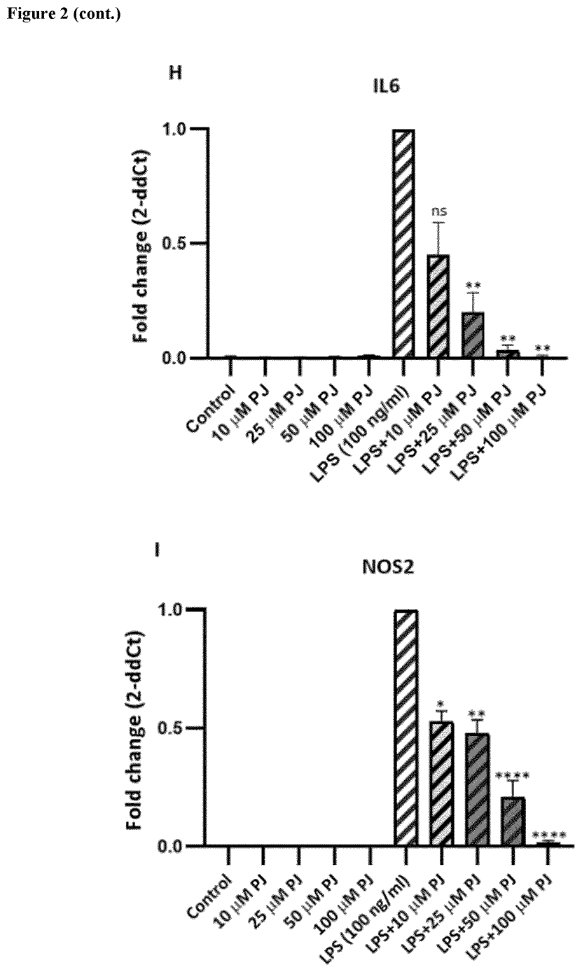

We decided to isolate three structurally related pectinolides, which only differ in one substituent at position 5 (R). This structural similarity was used to explore the structure-activity relationship of these compounds. Specifically, Hyp contains a Hydrogen (R=H), Pectinolide E contains an acetoxy group (R=OAc), and Pectinolide J contains a hydroxyl group (R=OH) (FIG. 1A).

### Example 2.2. Molecular Docking of the Compounds of the Invention on Key Innate Immune Targets

In order to investigate the potential biological activity of the compounds of the invention, we decided to run a docking analysis test against key inflammatory targets (TLR4, IL1R, COX1, and COX2) (FIG. 1B). Docking scores obtained, represented as kcal/mol, revealed a strong bind with the studied targets (Table 5).

For TLR4 receptor, docking scores of the compounds of the invention were stronger than the one of LPS (a widely known TLR4 agonist) (Table 5). In comparison to HYP, PE and PJ dockings were slightly greater towards this receptor. This difference was not observed between PE and PJ (Table 5). The docking analysis was also run against IL1R, another innate immune receptor. In this case, docking scores of the compounds of the invention were similar to Methotrexate (a known IL1R inhibitor). As observed for TLR4, PE and PJ exhibited the highest docking scores, which were significantly greater than HYP docking score (Table 5).

Considering the key role of COX enzymes activity in OA inflammatory responses, we further investigated the docking ability of the compounds of the invention against them. Data obtained showed that unlike HYP PE and PJ docking scores were stronger than indomethacin (a well-known COX1/2). To note, Hyp also exhibited a notable docking score against these enzymes.

TABLE 5

| (Kcal/mol) | TLR4 | IL1R | COX1 | COX2 |
|---|---|---|---|---|
| Hyptolide | −7.0 | −5.5 | −5.7 | −7.1 |
| Pectinolide E | −7.3 | −6.4 | −9.2 | −10.1 |
| Pectinolide J | −7.3 | −6.3 | −9.1 | −11.2 |
| A well-known agonist | −5.8 | −6.7 | −7.7 | −9.6 |

### Example 2.3. Effect of the Compounds of the Invention on Chondrocytes Viability

Aimed to evaluate the potential anti-inflammatory effects of the compounds of the invention without any toxic effect, we evaluated through a 48 h MTT assay whether the isolated compounds affected cell viability. As shown in FIG. 1 C-H, the compounds alone or in combination with inflammatory stimuli (LPS or IL1β) did not significantly affect ATDC5 cell viability.

### Example 2.4. The Compounds of the Invention Suppress NO Production in the ATDC5 Cell Line

NO is a key inflammatory mediator released upon TLR4 and IL-1R activation. Therefore, to evaluate the potential anti-TLR4 and -IL1R of the compounds of the invention ATDC5 cells were stimulated for 24 h with LPS (100 ng/ml) or IL1B (0.5 ng/ml) in the presence or absence of the compounds of the invention. As shown in FIG. 1, LPS and IL1B increased NO production, determined as nitrite accumulation. In contrast, the compounds of the invention alone did not modulate NO production. However, these compounds were able to significantly reduce NO production induced by LPS and IL1β in a dose-response fashion (FIG. 1 I-K). The activity of the compounds of the invention was not even reducing NO production. Specifically, Pectinolide J (R=OH) compound exhibited the maximum inhibitory activity (FIG. 1K) [10-25 μM]. Nonetheless, all the compounds of the invention significantly reduced the induced NO production above a certain concentration threshold (50 μM).

### Example 2.5. The Compounds of the Invention Suppress the Expression of Proinflammatory Mediators Upon TLR4 Activation

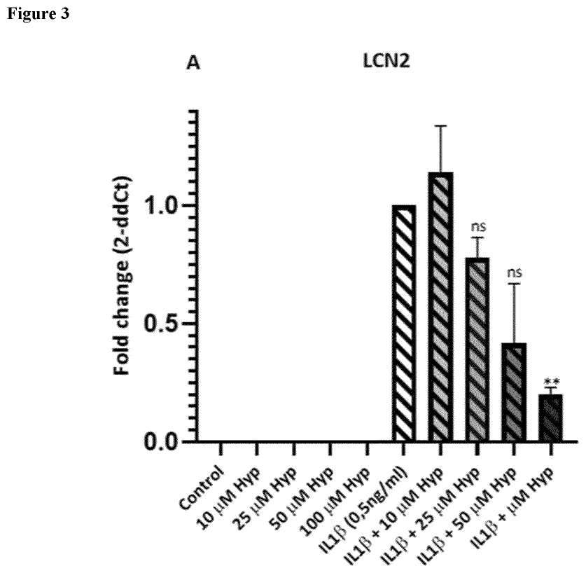

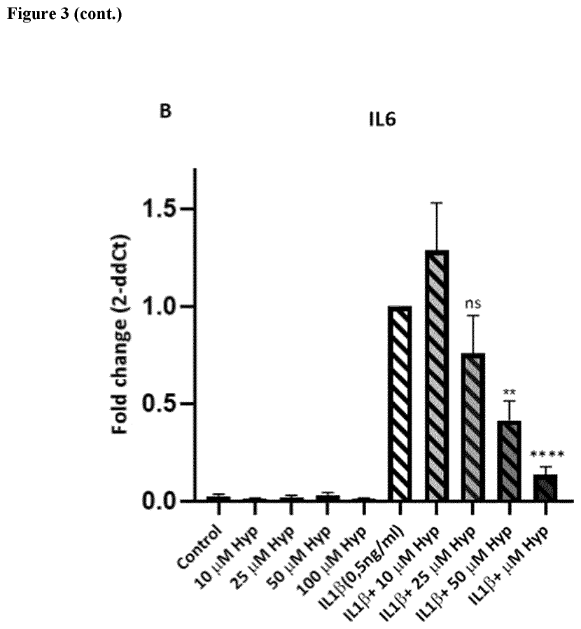

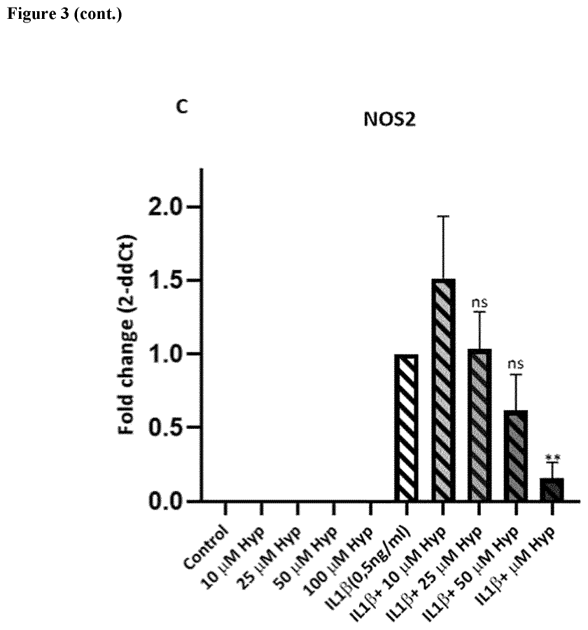

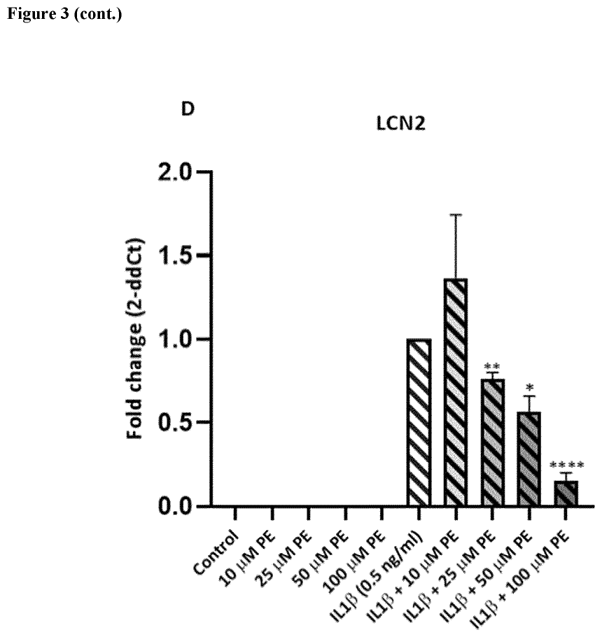

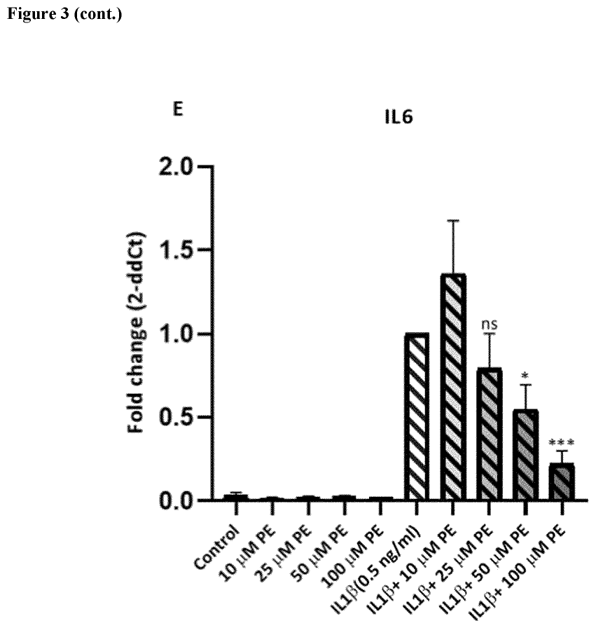

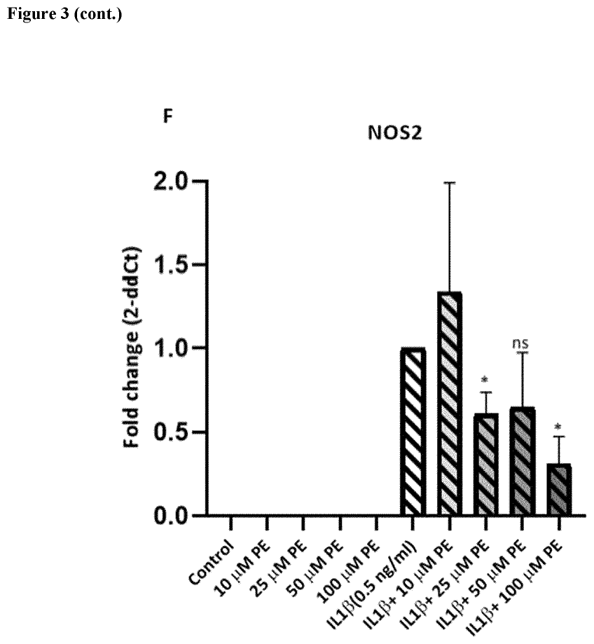

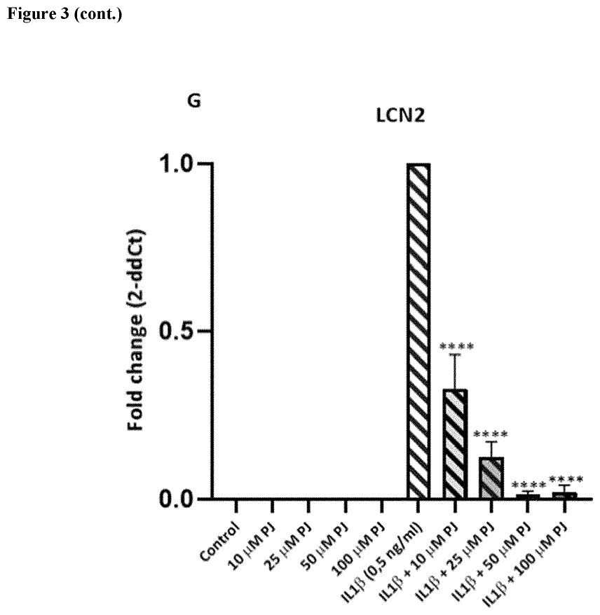

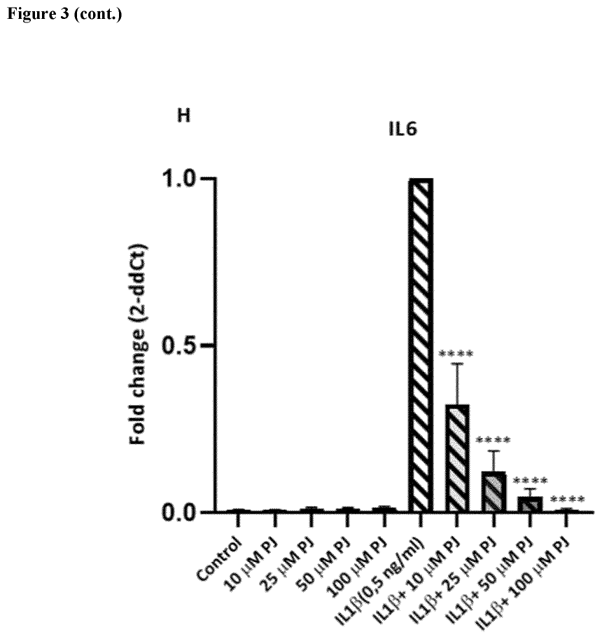

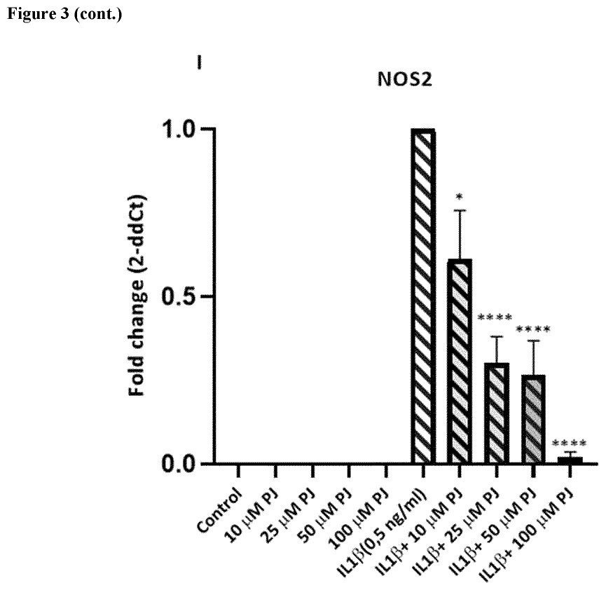

Activation of toll-like receptor 4 (TLR4) is associated with innate immune responses (IIRs) in articular tissues (23). Among these responses, TLR4 activation enhances the expression of pro-inflammatory mediators such as lipocalin-2 (LCN2), interleukin-6 (IL6) and nitric oxide synthase 2 (NOS2). To elucidate the anti-TLR4 activity of the compounds of the invention, we examined the LPS-induced mRNA expression of these mediators in the presence of the compounds of the invention. These compounds alone did not modify the expression of LCN2, IL6 and NOS2 after 24 h stimulation. However, as shown in FIG. 3, the expression of LPS-induced IIRs was significantly decreased upon treatment with the compounds of the invention. Above a certain concentration (25 μM) all the compounds were able to significantly decrease the mRNA expression of the inflammatory mediators. Remarkably, PJ compound exhibited the highest anti-inflammatory activity, showing similar results at lower concentrations.

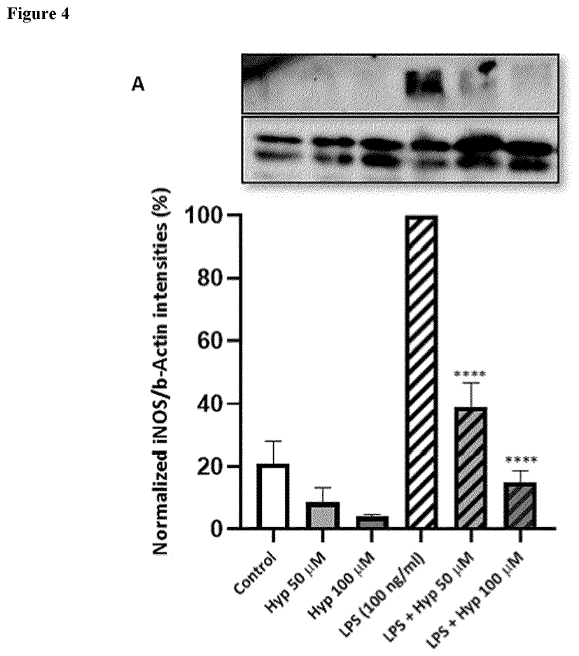

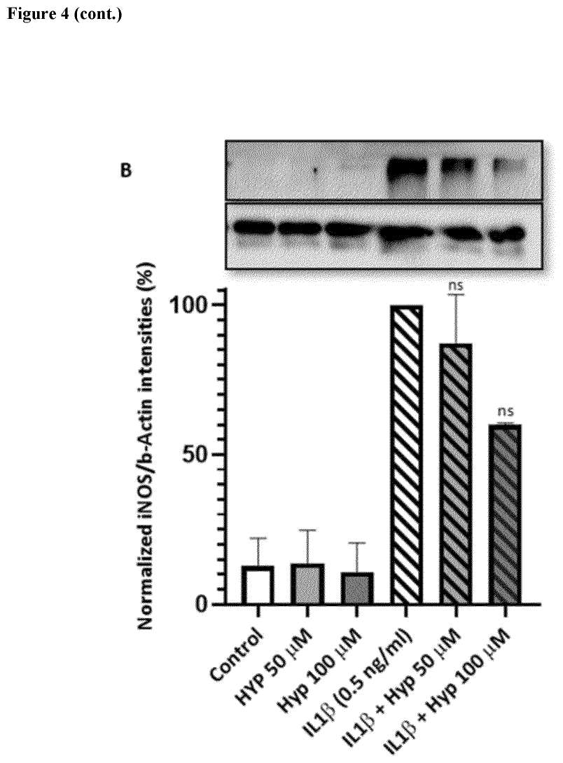

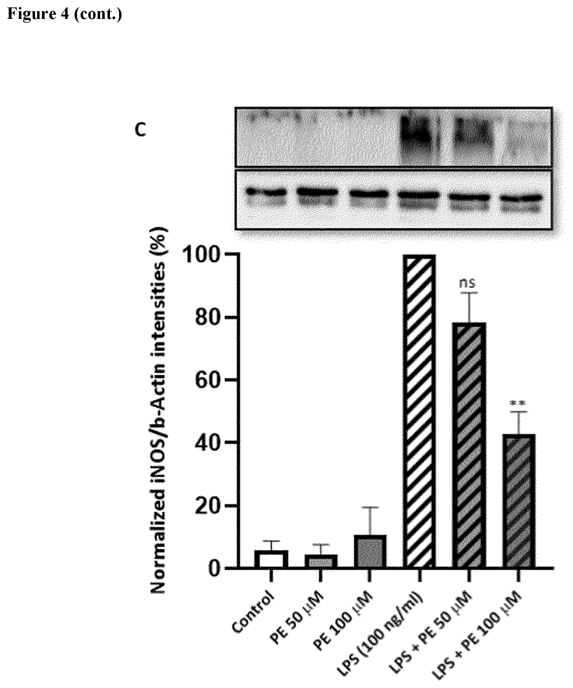

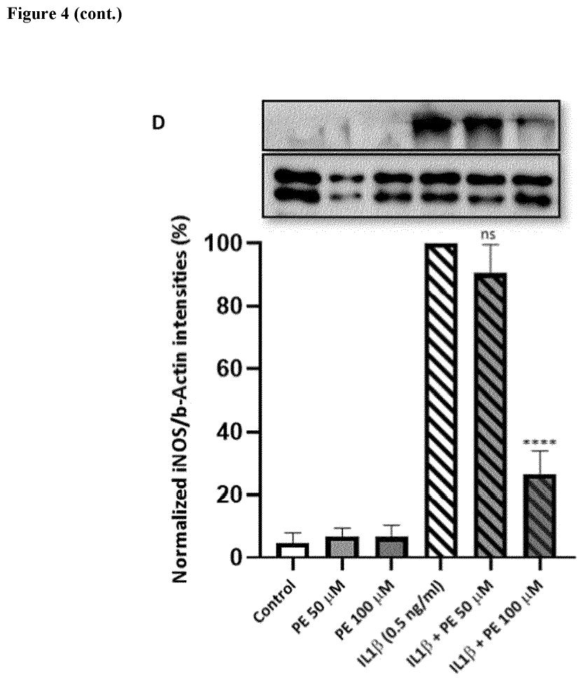

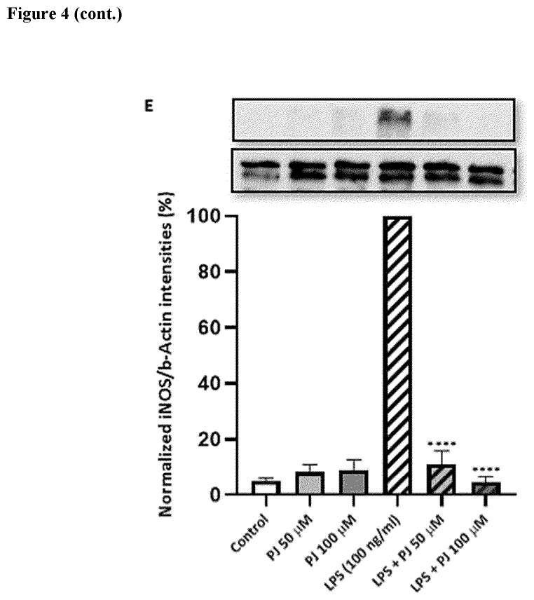

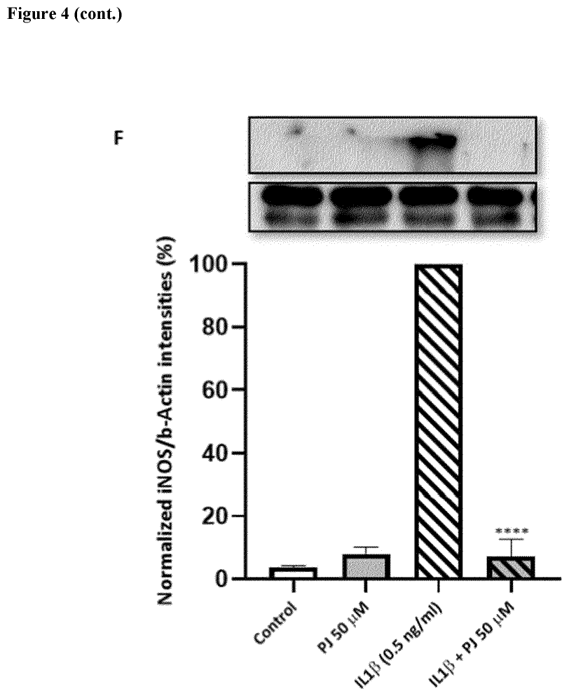

To further validate this result, we evaluated the protein expression of the 250 kDa NOS2 dimer, which is an enzyme highly induced upon TLR4- and IL1R-activation. As shown in FIG. 4, LPS stimulation induced NOS2 expression in ATDC5 cells. However, this expression was significantly decreased after co-stimulation with the compounds of the invention. Consistent with previous experiments, PJ induced the highest reduction of NOS2 expression (FIG. 4 E).

### Example 2.6. The Compounds of the Invention Suppress the Expression of Inflammatory Mediators Upon IL1R Activation

Activation of interleukin-1 receptor (IL1R) is associated with innate immune responses (IIRs) in articular tissues (23). Like TLR4 activation, IL1R activation enhances the expression of LCN2, IL6 NOS2. To elucidate the anti-IL1 activity of the compounds of the invention, we examined the IL1β-induced mRNA expression of these mediators in the presence of the compounds of the invention. After 24 h stimulation, these compounds alone did not modify the expression of any of the IIRs studied. However, as shown in FIG. 3, the IL1β-induced expression of LCN2, IL6 and NOS2 was significantly decreased upon treatment with the compounds of the invention. As observed in LPS-treated cells, PJ compound exhibited the maximum inhibitory activity (FIG. 3A-I) [10 μM] followed by PE [50 μM] and Hyp [100 μM]. Nonetheless, above a certain concentration threshold (25 μM) all the compounds significantly decrease the mRNA expression of several inflammatory mediators. As performed in the LPS-stimulated ATDC5 cells, to further validate these results we evaluated the protein expression of the 250 kDa NOS2 dimer. As shown in FIG. 4, IL1β stimulation induced NOS2 expression in ATDC5 cells. However, this expression was significantly decreased after co-stimulation with the compounds of the invention. Similarly, to the effects of PJ on LPS-stimulated cells, it induced the highest reduction of NOS2 expression among the tested compounds (FIG. 4 F).

### Example 2.7. The Compounds of the Invention Reduce the COX 1/2 Enzymatic Activity

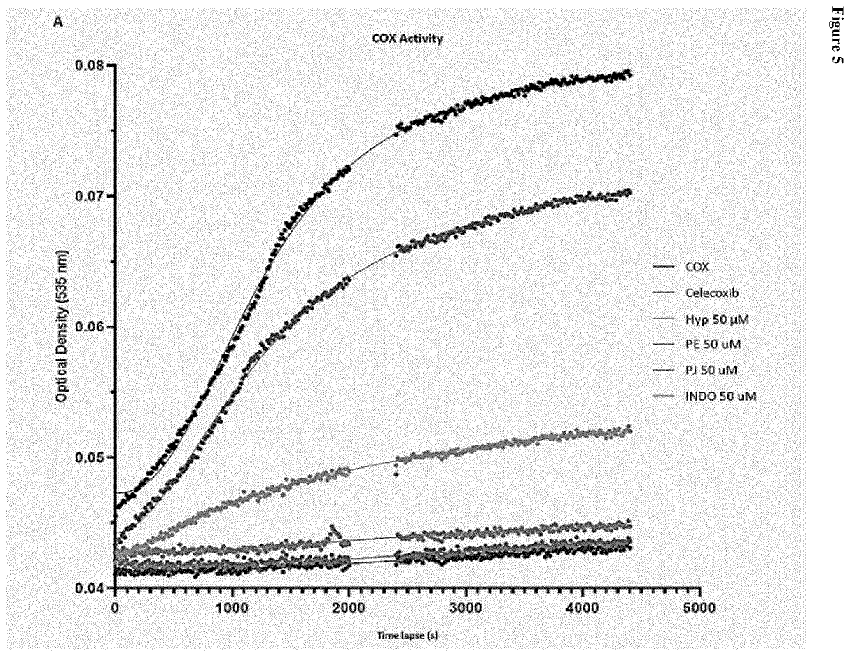

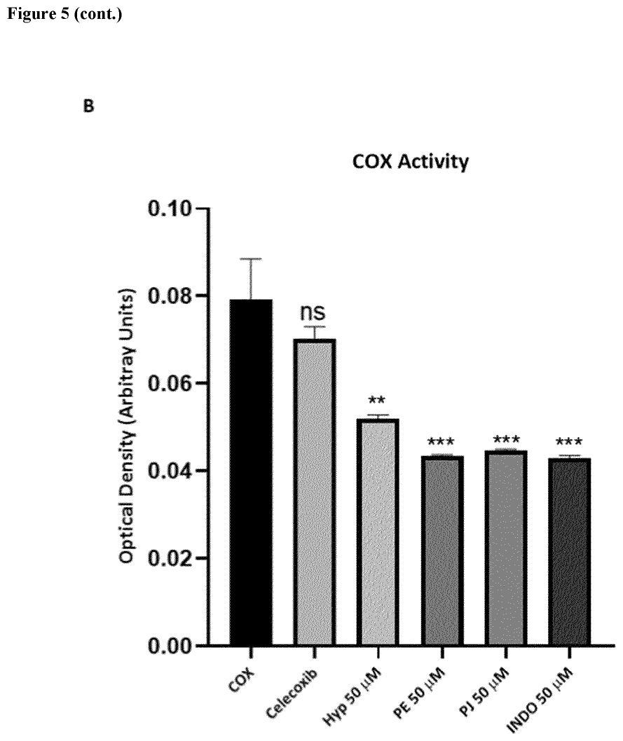

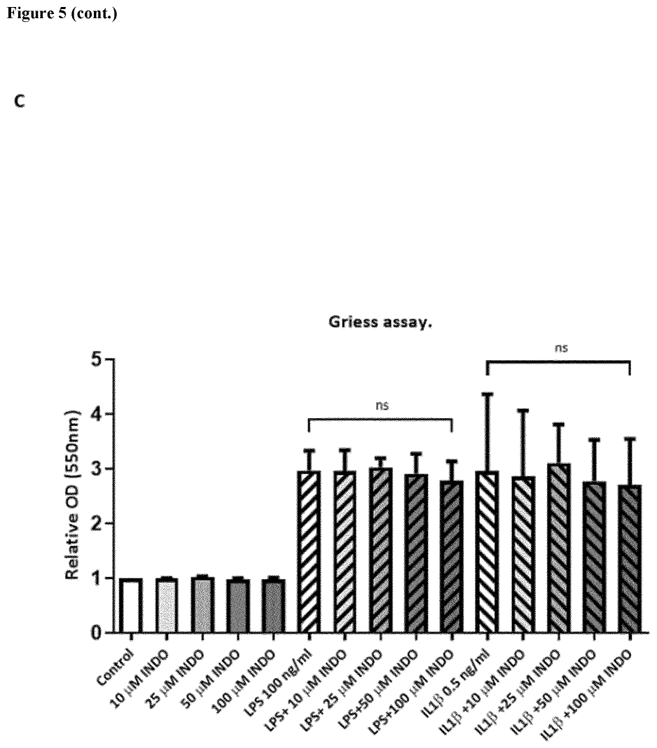

Cyclooxygenases (COX 1/2) are enzymes highly involved in articular inflammatory processes through their prostaglandin synthase activity. According to the molecular docking results, the compounds of the invention may inhibit these enzymes. Therefore, using a resorufin-based assay, we explored COX 1/2 activity in the presence or absence of the compounds of the invention. As shown in the FIG. 5, the compounds of the invention significantly reduced COX 1/2-mediated resorufin production. Unlike for Hyp, this effect was comparable to indomethacin activity, a well-known (COX 1/2) inhibitor. To elucidate whether the compounds of the invention show both activities (anti-COX1 and anti-COX2) we also compared the results obtained with the compounds of the invention with the specific COX2 inhibitor (Celecoxib). Consistent with a dual action on COX1/2 enzymes, the inhibitory activity of the compounds of the invention was greater than celecoxib.

According to these results, we decided to investigate if indomethacin also showed anti-TLR4 and anti-IL1R activity on LPS treated chondrocytes. Indomethacin did not significantly inhibit TLR4 nor IL1R-mediated inflammatory responses, suggesting that the blockade of these receptors by the compounds of the invention was independent of their anti-COX1/2 activity.

### Example 2.8. The Compounds of the Invention Suppress the Pro-Inflammatory Protein RBP4

RBP4 has been described as an adipokine that has been associated with several metabolic disorders such as diabetes, obesity, or metabolic syndrome, it has also been given a relevant role in other diseases characterised by a chronic inflammatory state like arthritis. Accordingly, previous reports have suggested that RBP4 might bind to Toll-like receptor 4 (TLR4). As shown in FIG. 6, Pectinolide J (R=OH) downregulates in chondrocytes RBP4 mRNA expression in both basal or inflammatory conditions generated by lipopolysaccharide (LPS) or interleukin 1 beta (IL1β). This is a relevant result because Pectinolide J, apart from blocking TLR4, could also reduce the positive inflammatory effect of RBP4 working as a TLR4 activating agent.

### Example 2.9. Validation Data of the Therapeutic Effect of the Compounds of the Invention in Primary Human Chondrocytes, Primary Human Osteoblasts and Human Synoviocytes

### Example 2.9.1. The Compounds of the Invention Blocked TLR4-IL1R-Mediated Innate Immune Responses in Primary Human Chondrocytes

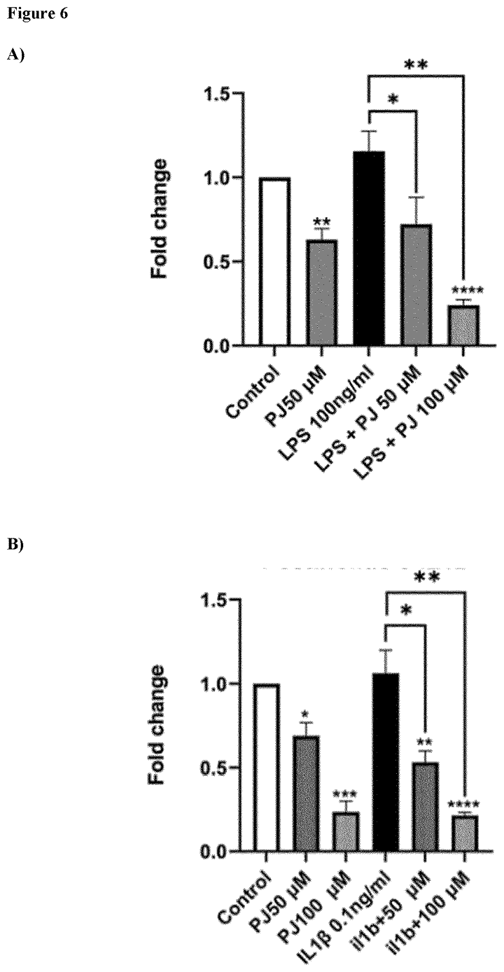

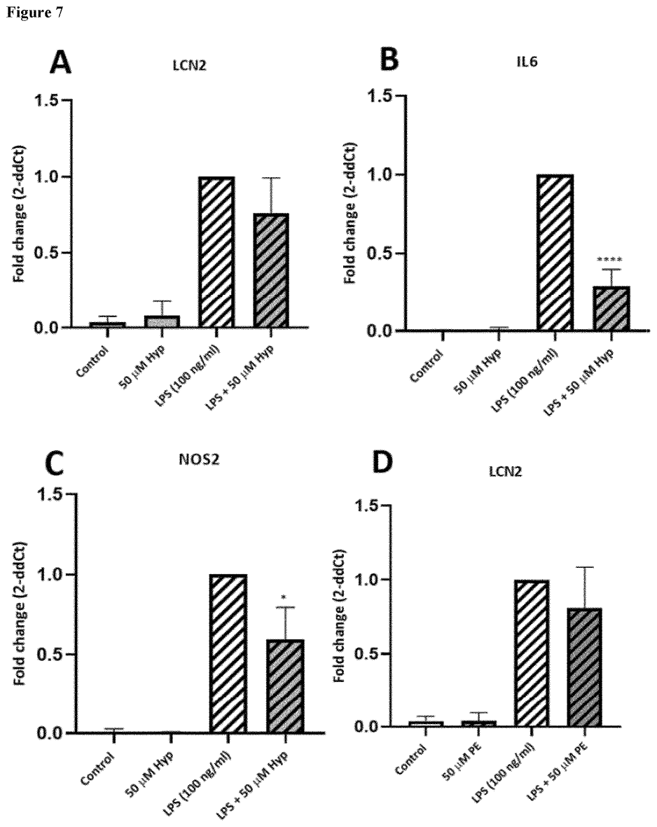

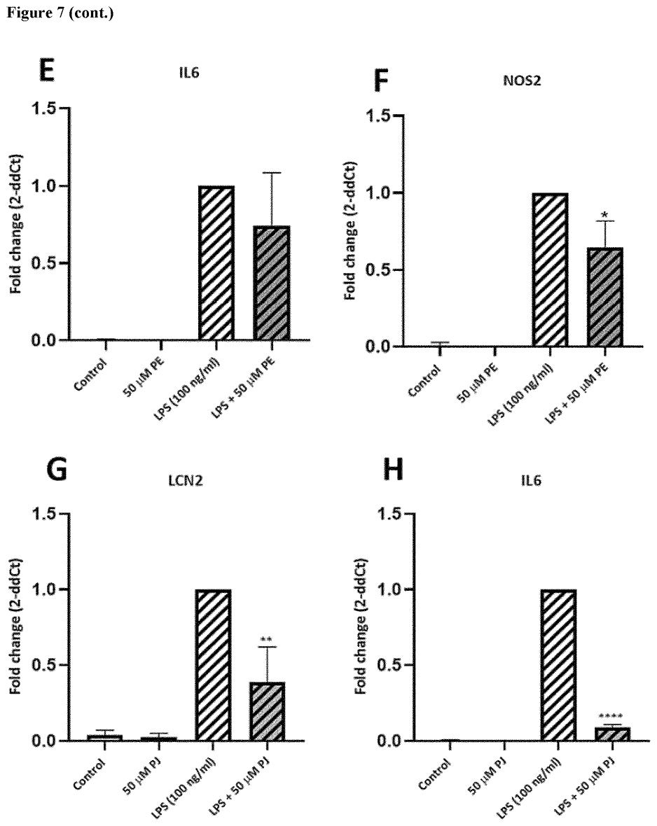

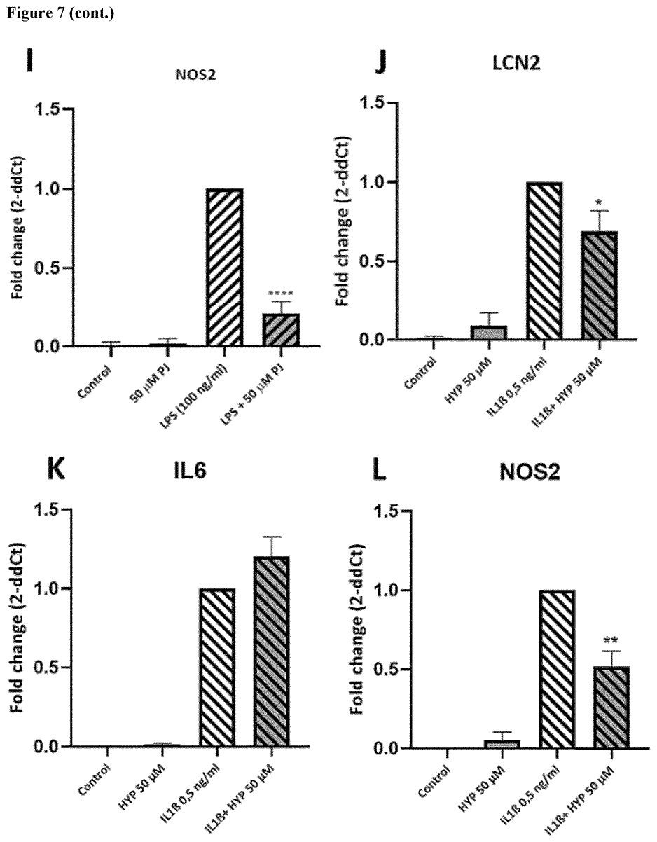

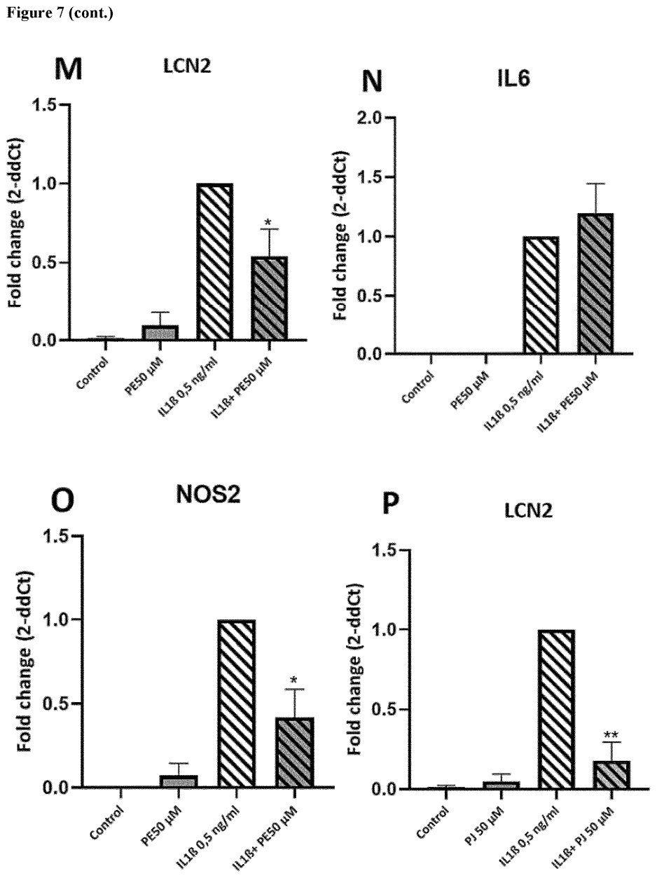

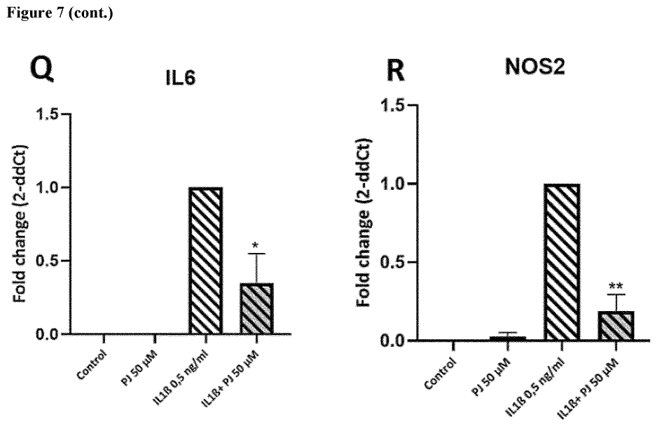

To validate ATDC5 results in a human context, we studied the effects of the studied compounds on TLR4- and IL1R-activated primary chondrocytes. We selected the concentration 50 μM and co-treated the primary cells with inflammatory stimulus for 24 h at the same concentrations [LPS (100 ng/ml) or IL1β (0.5 ng/ml)]. Consistent with the results obtained in ATDC5 cells, the inflammatory and catabolic markers genes induced by the inflammatory stimuli (LCN2, NOS2, IL6) were significantly reduced by the compounds of the invention (FIG. 7). As revealed by ATDC5 experiments, Pectinolide J showed the maximum anti-inflammatory activity (FIGS. 7 G-I and P-R).

### Example 2.9.2. The Compounds of the Invention Blocked TLR4-IL1R-Mediated Innate Immune Responses in Primary Human Osteoblasts

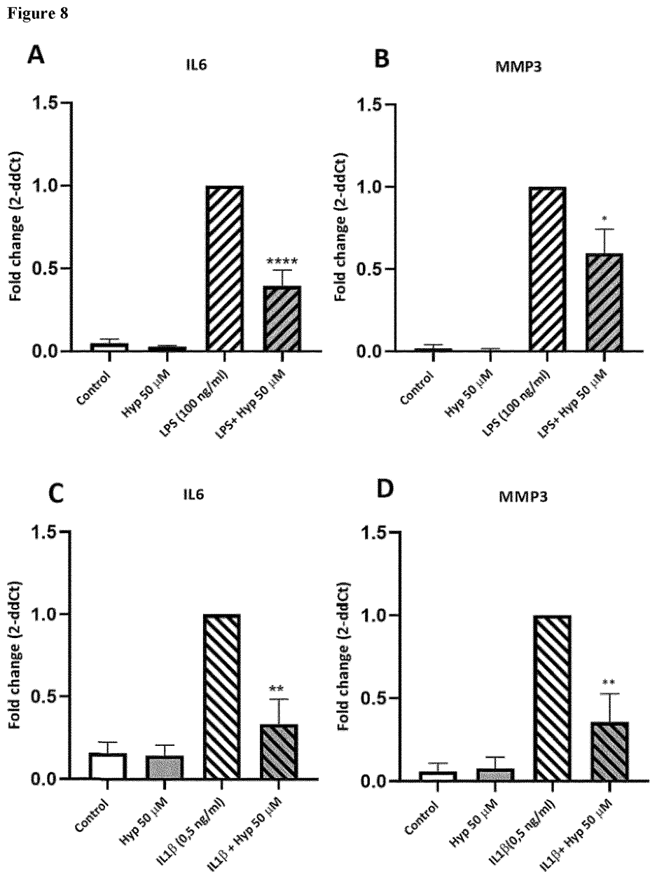

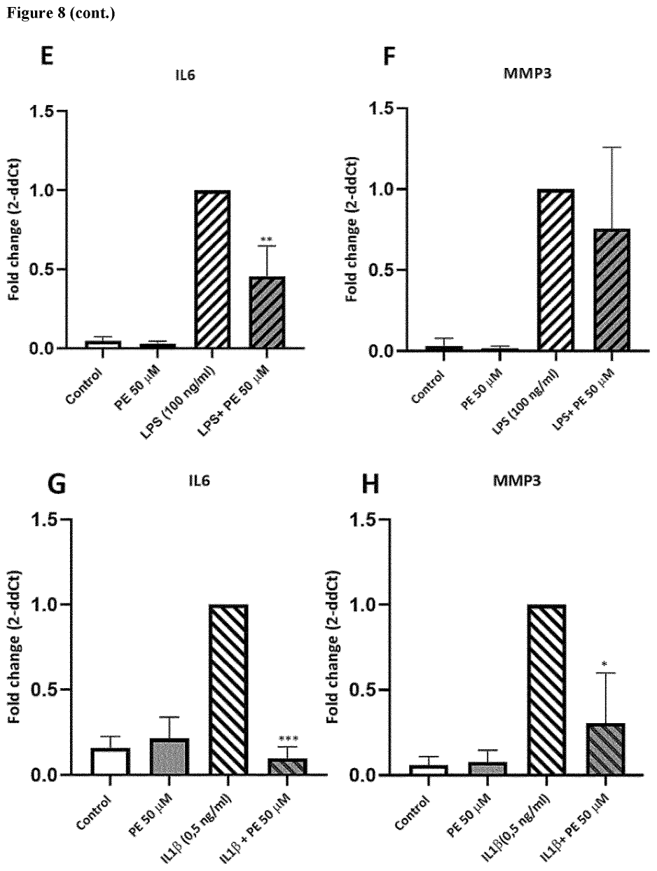

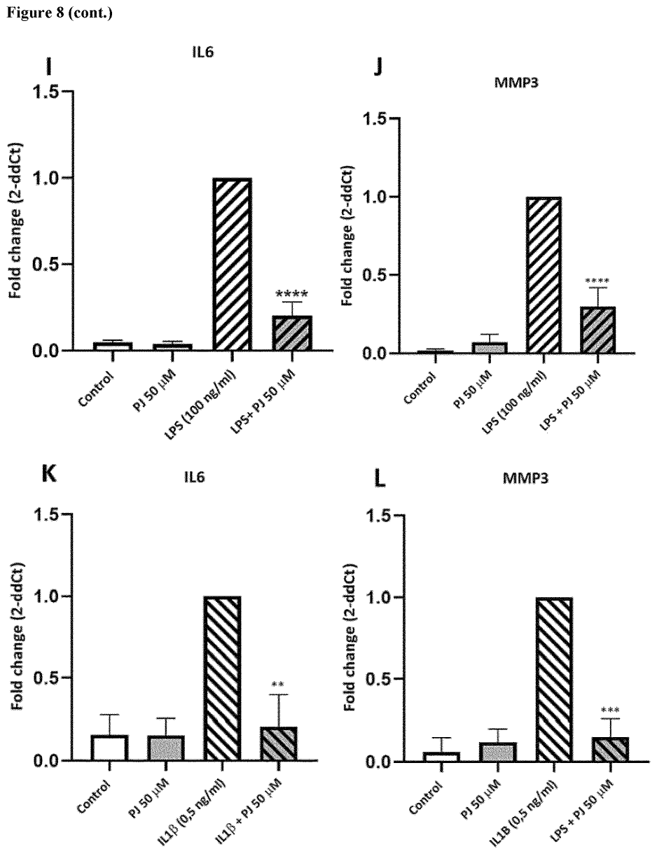

Furthermore, the effects of the compounds of the invention were studied on TLR4- and IL1R-activated primary osteoblasts. According to the results obtained in ATDC5 cells, we selected the concentration 50 μM and co-treated the primary cells with inflammatory stimuli for 24 h at the same concentrations [LPS (100 ng/ml), and IL1B (0.5 ng/ml)]. Consistent with the results obtained primary chondrocytes, after 24 h treatment with the studied compounds, the inflammatory and catabolic markers genes induced by the inflammatory stimuli (LCN2, NOS2, IL6) were significantly reduced in osteoblast primary cultures (FIG. 8).

### Example 2.9.3. The Compounds of the Invention Blocked TLR4-IL1R-Mediated Innate Immune Responses in Human Synoviocytes

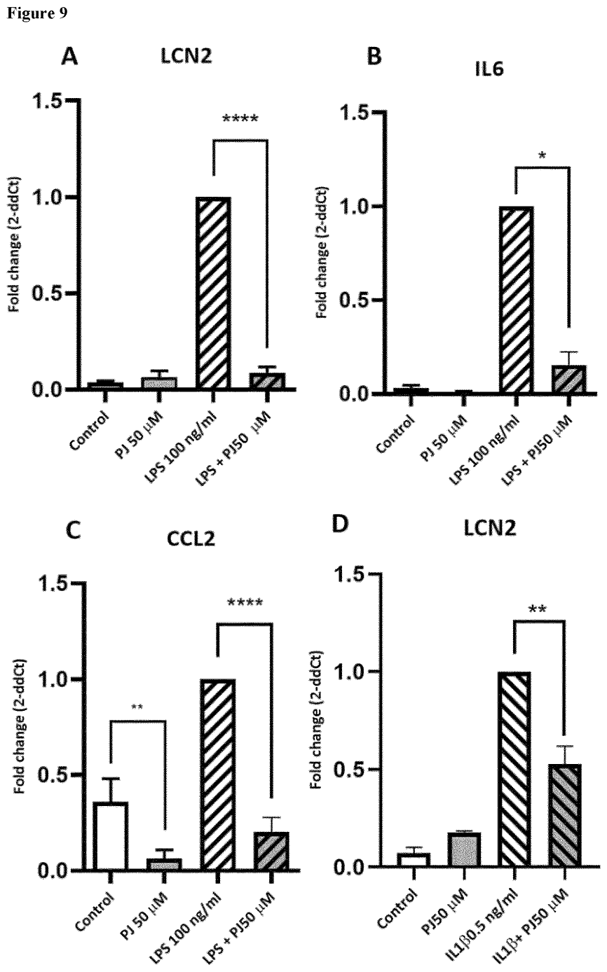

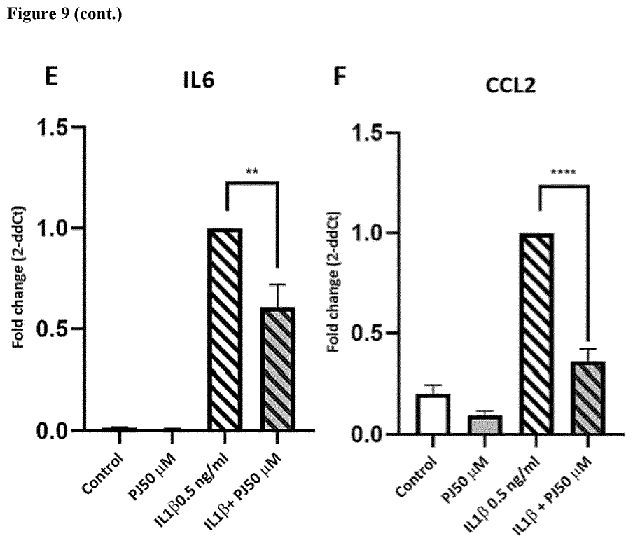

In order to validate the effectiveness of the compounds of the invention in synoviocytes, we selected Pectinolide J as a representative compound, and we studied the anti-inflammatory activity of this compound in human synoviocytes. Consistent with this, Pectinolide J significantly reduced the inflammatory and catabolic markers genes (LCN2, IL6 and CCL2, FIG. 9).

### REFERENCES

1. Blokhuis T J, Frölke J P M. Is radiation superior to indomethacin to prevent heterotopic ossification in acetabular fractures?: a systematic review. Clin Orthop Relat Res [Internet]. 2009 [cited 2023 Jan. 19]; 467 (2): 526-30. Available from: https://pubmed.ncbi.nlm.nih.gov/18820982/2.
2. Emrick J J, Mathur A, Wei J, Gracheva E O, Gronert K, Rosenblum M D, et al. Tissue-specific contributions of Tmem79 to atopic dermatitis and mast cell-mediated histaminergic itch. Proc Natl Acad Sci USA [Internet]. 2018 Dec. 18 [cited 2023 Jul. 12]; 115 (51): E12091-100. Available from: https://pubmed.ncbi.nlm.nih.gov/30463955/3.
3. Joice M, Vasileiadis G I, Amanatullah D F. Non-steroidal anti-inflammatory drugs for heterotopic ossification prophylaxis after total hip arthroplasty: a systematic review and meta-analysis. Bone Joint J [Internet]. 2018 Jul. 1 [cited 2023 Jul. 13]; 100-B (7): 915-22. Available from: https://pubmed.ncbi.nlm.nih.gov/29954195/4.
4. Brunnekreef J J, Hoogervorst P, Ploegmakers M J, Rijnen W H, Schreurs B W. Is etoricoxib effective in preventing heterotopic ossification after primary total hip arthroplasty? Int Orthop [Internet]. 2013 April [cited 2023 Jul. 13]; 37 (4): 583-7. Available from: https://pubmed.ncbi.nlm.nih.gov/23359100/5.
5. Katori M, Majima M. Cyclooxygenase-2: its rich diversity of roles and possible application of its selective inhibitors. Inflamm Res [Internet]. 2000 [cited 2023 Jul. 13]; 49 (8): 367-92. Available from: https://pubmed.ncbi.nlm.nih.gov/11028754/6.
6. Van Ryn J, Pairet M. Clinical experience with cyclooxygenase-2 inhibitors. Inflamm Res [Internet]. 1999 [cited 2023 Jul. 13]; 48 (5): 247-54. Available from: https://pubmed.ncbi.nlm.nih.gov/10391112/7.
7. DiCesare P E, Nimni M E, Peng L, Yazdi M, Cheung D T. Effects of indomethacin on demineralized bone-induced heterotopic ossification in the rat. J Orthop Res [Internet]. 1991 [cited 2023 Jul. 13]; 9 (6): 855-61. Available from: https://pubmed.ncbi.nlm.nih.gov/1919848/8.
8. Malcova H, Strizova Z, Milota T, Striz I, Sediva A, Cebecauerova D, et al. IL-1 Inhibitors in the Treatment of Monogenic Periodic Fever Syndromes: From the Past to the Future Perspectives. Front Immunol [Internet]. 2021 Feb. 1 [cited 2023 Jan. 17]; 11. Available from: https://pubmed.ncbi.nlm.nih.gov/33603750/WO
9. Bodar E J, Kuijk L M, Drenth J P H, Van Der Meer J W M, Simon A, Frenkel J. On-demand anakinra treatment is effective in mevalonate kinase deficiency. Ann Rheum Dis [Internet]. 2011 December [cited 2023 Jun. 19]; 70 (12): 2155-8. Available from: https://pubmed.ncbi.nlm.nih.gov/21859689/10.
Dinarello C A. Treating inflammation by blocking interleukin-1 in humans. 2011; 4 (164): 469-84.
11. Malemud C J. Matrix Metalloproteinases and Synovial Joint Pathology [Internet]. 1st ed. Vol. 148, Progress in Molecular Biology and Translational Science. Elsevier Inc.; 2017. 305-325 p. Available from: http://dx.doi.org/10.1016/bs.pmbts.2017.03.003
12. Int J. Pergamon CYTOKINES IN HIV INFECTION. 1994; 16 (5): 391-6.
13. Kedzierska K, Crowe S M. Cytokines and HIV-1: Interactions and clinical implications. Antivir Chem Chemother. 2001; 12 (3): 133-50.
14. Yaseen M M, Abuharfeil N M, Darmani H. The role of IL-1β during human immunodeficiency virus type 1 infection. Rev Med Virol [Internet]. 2022 [cited 2023 Jan. 17]; Available from: https://pubmed.ncbi.nlm.nih.gov/36209388/15.
15. Blackwell T S, Christman J W. Sepsis and cytokines: Current status. Br J Anaesth. 1996; 77 (1): 110-7.
16. Saag K G, Khanna P P, Keenan R T, Ohlman S, Osterling Koskinen L, Sparve E, et al. A Randomized, Phase II Study Evaluating the Efficacy and Safety of Anakinra in the Treatment of Gout Flares. Arthritis Rheumatol (Hoboken, NJ) [Internet]. 2021 Aug. 1 [cited 2023 Jun. 19]; 73 (8): 1533-42. Available from: https://pubmed.ncbi.nlm.nih.gov/33605029/17.
17. Dinarello C A. The IL-1 family of cytokines and receptors in rheumatic diseases. Nat Rev Rheumatol [Internet]. 2019; 15 (10): 612-32. Available from: http://dx.doi.org/10.1038/s41584-019-0277-8
18. Na H S, Park J S, Cho K H, Kwon J Y, Choi J W, Jhun J, et al. Interleukin-1-Interleukin-17 Signaling Axis Induces Cartilage Destruction and Promotes Experimental Osteoarthritis. Front Immunol [Internet]. 2020 May 5 [cited 2023 Jun. 19]; 11. Available from: https://pubmed.ncbi.nlm.nih.gov/32431699/19.
19. Nishimura R, Hata K, Takahata Y, Murakami T, Nakamura E, Ohkawa M, et al. Role of Signal Transduction Pathways and Transcription Factors in Cartilage and Joint Diseases. Int J Mol Sci [Internet]. 2020 Feb. 2 [cited 2023 Jun. 19]; 21 (4). Available from: https://pubmed.ncbi.nlm.nih.gov/32079226/20.
20. Thilak J, Panakkal J J, Kim T Y, Goodman S M, Lee S S, Salvati E A. Risk Factors of Heterotopic Ossification Following Total Hip Arthroplasty in Patients With Ankylosing Spondylitis. J Arthroplasty [Internet]. 2015 Dec. 1 [cited 2022 Mar. 11]; 30 (12): 2304-7. Available from: https://pubmed.ncbi.nlm.nih.gov/26154570/21.
21. Tseng H W, Kulina I, Girard D, Gueguen J, Vaquette C, Salga M, et al. Interleukin-1 Is Overexpressed in Injured Muscles Following Spinal Cord Injury and Promotes Neurogenic Heterotopic Ossification. J Bone Miner Res [Internet]. 2021 [cited 2022 Mar. 11]; Available from: https://pubmed.ncbi.nlm.nih.gov/34841579/22.
22. Tseng H W, Kulina I, Girard D, Gueguen J, Vaquette C, Salga M, et al. Interleukin-1 Is Overexpressed in Injured Muscles Following Spinal Cord Injury and Promotes Neurogenic Heterotopic Ossification. J Bone Miner Res [Internet]. 2022 Mar. 1 [cited 2023 Jun. 19]; 37 (3): 531-46. Available from: https://pubmed.ncbi.nlm.nih.gov/34841579/23.
23. Gómez R, Villalvilla A, Largo R, Gualillo O, Herrero-Beaumont G. TLR4 signalling in osteoarthritis-finding targets for candidate DMOADs. Nat Rev Rheumatol [Internet]. 2015 Mar. 16 [cited 2019 Jun. 26]; 11 (3): 159-70. Available from: http://www.nature.com/articles/nrrheum.2014.209
24. Alonso-Pérez A, Franco-Trepat E, Guillán-Fresco M, Jorge-Mora A, López V, Pino J, et al. Role of toll-like receptor 4 on osteoblast metabolism and function. Front Physiol. 2018; 9 (MAY): 1-12.
25. Physiology C. Amitriptyline Reduces Inflammation and Mortality in a Murine Model of Sepsis. Cell Physiol Biochem. 2019; 52 (3): 565-79.
26. McCormack W J, Parker A E, O'Neill L A. Toll-like receptors and NOD-like receptors in rheumatic diseases. Arthritis Res Ther [Internet]. 2009 Oct. 14 [cited 2023 Jan. 12]; 11 (5). Available from: https://pubmed.ncbi.nlm.nih.gov/19835640/27.
27. Franco-Trepat E, Alonso-Pérez A, Guillán-Fresco M, Jorge-Mora A, Crespo-Golmar A, López-Fagúndez M, et al. Amitriptyline blocks innate immune responses mediated by toll-like receptor 4 and IL-1 receptor: Preclinical and clinical evidence in osteoarthritis and gout. Br J Pharmacol. 2022; 179 (2): 270-86.
28. Sun X, Li P, Qu X, Liu W. Isovitexin alleviates acute gouty arthritis in rats by inhibiting inflammation via the TLR4/MyD88/NF-κB pathway. Pharm Biol [Internet]. 2021 [cited 2023 Jan. 12]; 59 (1): 1326-33. Available from: https://pubmed.ncbi.nlm.nih.gov/34582722/29.
29. Dang W tai, Xu D, Zhou J guo. Effect of Berberine on Activation of TLR4-NFκB Signaling Pathway and NLRP3 Inflammasome in Patients with Gout. Chin J Integr Med [Internet]. 2023 Jan. 1 [cited 2023 Jan. 12]; 29 (1). Available from: https://pubmed.ncbi.nlm.nih.gov/36125615/30.
30. Mitchell E J, Canter J, Norris P, Jenkins J, Morris J. The genetics of heterotopic ossification: Insight into the bone remodeling pathway. J Orthop Trauma [Internet]. 2010 September [cited 2020 Feb. 11]; 24 (9): 530-3. Available from: http://www.ncbi.nlm.nih.gov/pubmed/20736788
31. Mitchell E J, Canter J, Norris P, Jenkins J, Morris J. The genetics of heterotopic ossification: insight into the bone remodeling pathway. J Orthop Trauma [Internet]. 2010 September [cited 2022 Mar. 11]; 24 (9): 530-3. Available from: https://pubmed.ncbi.nlm.nih.gov/20736788/32.
32. Barruet E, Morales B M, Cain C J, Ton A N, Wentworth K L, Chan T V., et al. NF-κB/MAPK activation underlies ACVR1-mediated inflammation in human heterotopic ossification. JCI insight. 2018; 3 (22): 1-20.
33. Smith R L, Hebert H L, Massey J, Bowes J, Marzo-Ortega H, Ho P, et al. Association of Toll-like receptor 4 (TLR4) with chronic plaque type psoriasis and psoriatic arthritis. Arch Dermatol Res. 2016 Apr. 1; 308 (3): 201-5.
34. Frommer K W, Schäffler A, Rehart S, Lehr A, Müller-Ladner U, Neumann E. Free fatty acids: potential proinflammatory mediators in rheumatic diseases. Ann Rheum Dis [Internet]. 2015 Jan. 1 [cited 2023 Jan. 12]; 74 (1): 303-10. Available from: https://pubmed.ncbi.nlm.nih.gov/24285492/35.
35. Elloumi N, Fakhfakh R, Ayadi L, Sellami K, Abida O, Ben Jmaa M, et al. The Increased Expression of Toll-Like Receptor 4 in Renal and Skin Lesions in Lupus Erythematosus. J Histochem Cytochem. 2017; 65 (7): 389-98.
36. Marques C P C, Maor Y, de Andrade M S, Rodrigues V P, Benatti B B. Possible evidence of systemic lupus erythematosus and periodontal disease association mediated by Toll-like receptors 2 and 4. Clin Exp Immunol. 2016; 183 (2): 187-92.
37. Marques C P C, Rodrigues V P, de Carvalho L C, Nichilatti L P, Franco M M, Patrício F J B, et al. Expression of Toll-like receptors 2 and 4 in the saliva of patients with systemic lupus erythematosus and chronic periodontitis. Clin Rheumatol. 2021; 40 (7): 2727-34.
38. Heterotopic bone in hip arthroplasties. Cemented versus noncemented—PubMed [Internet]. [cited 2022 Mar. 11]. Available from: https://pubmed.ncbi.nlm.nih.gov/1516305/39.
39. Zhou L, Gee S M, Hansen J A, Posner M A. Heterotopic Ossification After Arthroscopic Procedures A Scoping Review of the Literature. Available from: http://www.sagepub.com/journals-permissions.
40. Roemer F W, Guermazi A, Niu J, Zhang Y, Mohr A, Felson D T. Prevalence of Magnetic Resonance Imaging-Defined Atrophic and Hypertrophic Phenotypes of Knee Osteoarthritis in a Population-Based Cohort. ARTHRITIS Rheum. 2012; 64 (2): 429-37.
41. Campolo M, Paterniti I, Siracusa R, Filippone A, Esposito E, Cuzzocrea S. TLR4 absence reduces neuroinflammation and inflammasome activation in Parkinson's diseases in vivo model. Brain Behav Immun. 2019; 76 (November): 236-47.
42. Yang L, Zhou R, Tong Y, Chen P, Shen Y, Miao S, et al. Neuroprotection by dihydrotestosterone in LPS-induced neuroinflammation. Neurobiol Dis [Internet]. 2020; 140:104814. Available from: https://doi.org/10.1016/j.nbd.2020.104814
43. Akamatsu Y, Pagan V A, Hanafy K A. The role of TLR4 and HO-1 in neuroinflammation after subarachnoid hemorrhage. J Neurosci Res. 2020; 98 (3): 549-56.
44. Khanmohammadi S, Rezaei N. Role of Toll-like receptors in the pathogenesis of COVID-19. J Med Virol [Internet]. 2021 May 1 [cited 2023 Jan. 12]; 93 (5): 2735-9. Available from: https://pubmed.ncbi.nlm.nih.gov/33506952/45.
45. Aboudounya M M, Heads R J. COVID-19 and Toll-Like Receptor 4 (TLR4): SARS-CoV-2 May Bind and Activate TLR4 to Increase ACE2 Expression, Facilitating Entry and Causing Hyperinflammation. Mediators Inflamm [Internet]. 2021 [cited 2023 Jan. 12]; 2021. Available from: https://pubmed.ncbi.nlm.nih.gov/33505220/46.
46. Kuzmich N N, Sivak K V., Chubarev V N, Porozov Y B, Savateeva-Lyubimova T N, Peri F. TLR4 Signaling Pathway Modulators as Potential Therapeutics in Inflammation and Sepsis. Vaccines [Internet]. 2017 Dec. 1 [cited 2023 Jan. 12]; 5 (4). Available from: https://pubmed.ncbi.nlm.nih.gov/28976923/47.
47. Browne E P. The role of toll-like receptors in retroviral infection. Microorganisms. 2020; 8 (11): 1-21.
48. Kyo F, Futani H, Matsui K, Terada M, Adachi K, Nagata K, et al. Endogenous interleukin-6, but not tumor necrosis factor alpha, contributes to the development of toll-like receptor 4/myeloid differentiation factor 88-mediated acute arthritis in mice. Arthritis Rheum [Internet]. 2005 August [cited 2023 Jul. 18]; 52 (8): 2530-40. Available from: https://pubmed.ncbi.nlm.nih.gov/16052598/49.
49. Eliçabe R J, Arias J L, Rabinovich G A, Di Genaro M S. TNFRp55 modulates IL-6 and nitric oxide responses following Yersinia lipopolysaccharide stimulation in peritoneal macrophages. Immunobiology. 2011; 216 (12): 1322-30.
50. Varoga D, Klostermeier E, Paulsen F, Wruck C, Lippross S, Brandenburg L O, et al. The antimicrobial peptide HBD-2 and the Toll-like receptors-2 and -4 are induced in synovial membranes in case of septic arthritis. Virchows Arch [Internet]. 2009 June [cited 2023 Jul. 18]; 454 (6): 685-94. Available from: https://pubmed.ncbi.nlm.nih.gov/19412702/51.
51. Zeng X zhou, Zhang Y yang, Yang Q, Wang S, Zou B hua, Tan Y hui, et al. Artesunate attenuates LPS-induced osteoclastogenesis by suppressing TLR4/TRAF6 and PLCγ1-Ca2+-NFATc1 signaling pathway. Acta Pharmacol Sin [Internet]. 2020 Feb. 1 [cited 2023 Jul. 18]; 41 (2): 229-36. Available from: https://pubmed.ncbi.nlm.nih.gov/31431733/52.
52. Liu C, Zhou X R, Ye M Y, Xu X Q, Zhang Y W, Liu H, et al. RBP4 Is Associated With Insulin Resistance in Hyperuricemia-Induced Rats and Patients With Hyperuricemia. Front Endocrinol (Lausanne) [Internet]. 2021 Jun. 10 [cited 2023 Jul. 13]; 12. Available from: https://pubmed.ncbi.nlm.nih.gov/34177800/WO
53. Retinol-binding protein 4 (RBP4) as the causative factor and marker of vascular injury related to insulin resistance-PubMed [Internet]. [cited 2023 Jul. 13]. Available from: https://pubmed.ncbi.nlm.nih.gov/28026829/54.
54. Domingos M A M, Queiroz M, Lotufo P A, Benseñor I J, Titan S M de O. Serum RBP4 and CKD: Association with insulin resistance and lipids. J Diabetes Complications [Internet]. 2017 Jul. 1 [cited 2023 Jul. 13]; 31 (7): 1132-8. Available from: https://pubmed.ncbi.nlm.nih.gov/28473187/55.
55. Li J Y, Chen X X, Lu X H, Zhang C B, Shi Q P, Feng L. Elevated RBP4 plasma levels were associated with diabetic retinopathy in type 2 diabetes. Biosci Rep [Internet]. 2018 Sep. 12 [cited 2023 Jul. 13]; 38 (5). Available from: https://pubmed.ncbi.nlm.nih.gov/30135138/56.
56. Kessel L, Bertelsen M, Grønskov K. RBP4-related eye disease in a Danish family with retinitis pigmentosa and congenital ocular malformations. Ophthalmic Genet [Internet]. 2022 [cited 2023 Jul. 13]; 43 (6): 876-81. Available from: https://pubmed.ncbi.nlm.nih.gov/36317196/57.
57. Li Z Z, Lu X Z, Liu J B, Chen L. Serum retinol-binding protein 4 levels in patients with diabetic retinopathy. J Int Med Res [Internet]. 2010 [cited 2023 Jan. 20]; 38 (1): 95-9. Available from: https://pubmed.ncbi.nlm.nih.gov/20233518/58.
58. Han W, Wei H, Kong W, Wang J, Yang L, Wu H. Association between retinol binding protein 4 and diabetic retinopathy among type 2 diabetic patients: a meta-analysis. Acta Diabetol [Internet]. 2020 Oct. 1 [cited 2023 Jan. 20]; 57 (10): 1203-18. Available from: https://pubmed.ncbi.nlm.nih.gov/32405713/59.
59. Francisco V, Sanz M J, Real J T, Marques P, Capuozzo M, Ait Eldjoudi D, et al. Adipokines in Non-Alcoholic Fatty Liver Disease: Are We on the Road toward New Biomarkers and Therapeutic Targets? Biology (Basel) [Internet]. 2022 Aug. 1 [cited 2023 Jul. 13]; 11 (8). Available from: https://pubmed.ncbi.nlm.nih.gov/36009862/60.
60. Huang H, Xu C. Retinol-binding protein-4 and nonalcoholic fatty liver disease. Chin Med J (Engl) [Internet]. 2022 May 20 [cited 2023 Jul. 13]; 135 (10): 1182-9. Available from: https://pubmed.ncbi.nlm.nih.gov/35787557/61.
61. Schiborn C, Weber D, Grune T, Biemann R, Jäger S, Neu N, et al. Retinol and Retinol Binding Protein 4 Levels and Cardiometabolic Disease Risk. Circ Res [Internet]. 2022 Sep. 16 [cited 2023 Jul. 13]; 131 (7): N. Available from: https://pubmed.ncbi.nlm.nih.gov/36017698/

1. Compound of Formula (I), or salts derived thereof, wherein OAc is an acetyloxy group and R is OH, H or OAc, 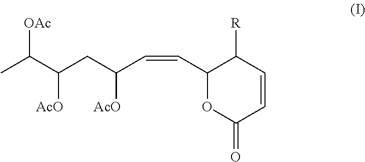
for use in the prevention and/or treatment of a disease selected from the group comprising: osteoarthritis, rheumatoid arthritis, ankylosing spondylitis, gout, pseudogout, heterotopic ossification, menstrual pain, atopic dermatitis, fibrodysplasia ossificans progressive (FOP), fever, tumour necrosis factor receptor-associated periodic syndrome (TRAPS), hyperimmunoglobulin D syndrome (HIDS), mevalonate kinase deficiency (MKD), human immunodeficiency virus (HIV) infection, sepsis, familial Mediterranean fever (FMF), septic arthritis, reactive arthritis, adult Still's disease, systemic juvenile idiopathic arthritis, COVID-19, cryopyrin-associated periodic syndromes (CAPS), gouty arthritis, psoriasis, psoriatic arthritis, inflammatory bowel disease (IBD), systemic lupus erythematosus (SLE), traumatic brain injury (TBI), neuroinflammation, influenza A virus (IAV) infection, respiratory syncytial virus (RSV) infection, dengue infection, Ebola virus disease or Chlamydia infection, insulin resistance, retinopathy, fatty liver disease or hypertension.

2. Compound of Formula (I) for use, according to claim 1, in the prevention and/or treatment of a disease mediated by the activity of TLR4, IL1R, COX1/2 and/or RBP4 in chondrocytes, osteoblasts and/or in synoviocytes.

3. Compound of Formula (I) for use, according to any of the claim 1 or 2, in the prevention and/or treatment of a disease selected from the group comprising: osteoarthritis, rheumatoid arthritis, ankylosing spondylitis, heterotopic ossification, adult Still's disease, systemic juvenile idiopathic arthritis, cryopyrin-associated periodic syndromes (CAPS), gouty arthritis, pseudogout, tumour necrosis factor receptor-associated periodic syndrome (TRAPS), familial Mediterranean fever (FMF), septic arthritis, reactive arthritis, fibrodysplasia ossificans progressive (FOP), psoriatic arthritis or systemic lupus erythematosus (SLE).

4. Compound of Formula (I) for use, according to any of the claims 1 to 3, wherein the cryopyrin-associated periodic syndrome (CAPS) is selected from the group comprising: Muckel-Wells syndrome, neonatal onset multisystem inflammatory disease (NOMID), chronic infantile neurological cutaneous and articular (CINCA) syndrome, familial cold autoinflammatory syndrome (FCAS) or familial cold urticaria (FCU).

5. Compound of Formula (I) for use, according to any of the claims 1 to 4, in the prevention and/or treatment of osteoarthritis.

6. Compound for use, according to any of the claims 1 to 5, wherein the compound is administered topically, orally or by means of an intraarticular, subcutaneous, intravenous or muscular injection.

7. Pharmaceutical composition comprising a compound of Formula (I), or salts derived thereof, wherein OAc is an acetyloxy group and R is OH, H or OAc, and, optionally, excipients or carriers pharmaceutically acceptable, for use in the prevention and/or treatment of a disease selected from the group comprising: osteoarthritis, rheumatoid arthritis, ankylosing spondylitis, gout, pseudogout, heterotopic ossification, menstrual pain, atopic dermatitis, fibrodysplasia ossificans progressive (FOP), fever, tumour necrosis factor receptor-associated periodic syndrome (TRAPS), hyperimmunoglobulin D syndrome (HIDS), mevalonate kinase deficiency (MKD), human immunodeficiency virus (HIV) infection, sepsis, familial Mediterranean fever (FMF), septic arthritis, reactive arthritis, adult Still's disease, systemic juvenile idiopathic arthritis, COVID-19, cryopyrin-associated periodic syndromes (CAPS), gouty arthritis, psoriasis, psoriatic arthritis, inflammatory bowel disease (IBD), systemic lupus erythematosus (SLE), traumatic brain injury (TBI), neuroinflammation, influenza A virus (IAV) infection, respiratory syncytial virus (RSV) infection, dengue infection, Ebola virus disease or Chlamydia infection, insulin resistance, retinopathy, fatty liver disease or hypertension.

8. Pharmaceutical composition for use, according to claim 7, in the prevention and/or treatment of a disease mediated by the activity of TLR4, IL1R, COX1/2 and/or RBP4 in chondrocytes, human osteoblasts and/or in synoviocytes.

9. Pharmaceutical composition for use, according to claim 7 or 8, in the prevention and/or treatment of a disease selected from the group comprising: osteoarthritis, rheumatoid arthritis, ankylosing spondylitis, heterotopic ossification, adult Still's disease, systemic juvenile idiopathic arthritis, cryopyrin-associated periodic syndromes (CAPS), gouty arthritis, pseudogout, tumour necrosis factor receptor-associated periodic syndrome (TRAPS), familial Mediterranean fever (FMF), septic arthritis, reactive arthritis, fibrodysplasia ossificans progressive (FOP), psoriatic arthritis or systemic lupus erythematosus (SLE).

10. Pharmaceutical composition for use, according to any of the claims 7 to 9, wherein the cryopyrin-associated periodic syndrome (CAPS) is selected from the group comprising: Muckel-Wells syndrome, neonatal onset multisystem inflammatory disease (NOMID), chronic infantile neurological cutaneous and articular (CINCA) syndrome, familial cold autoinflammatory syndrome (FCAS) or familial cold urticaria (FCU).

11. Pharmaceutical composition for use, according to any of the claims 7 to 10, in the prevention and/or treatment of osteoarthritis.

12. Pharmaceutical composition for use, according to any of the claims 7 to 11, wherein the composition is administered topically, orally or by means of an intraarticular, subcutaneous, intravenous or muscular injection.
